(19) United States
(12) Patent Application Publication
(10) Pub. No.: US 2026/0198902 A1
(43) Pub. Date: Jul. 16, 2026

(54) ULTRASONIC DIAGNOSTIC APPARATUS AND CONTROL METHOD THEREOF

(71) Applicant: SAMSUNG MEDISON CO., LTD., Hongcheon-gun (KR)

(72) Inventors: Soyeon KIM, Hongcheon-gun (KR); Soobin CHOI, Hongcheon-gun (KR); Jiyun KIM, Hongcheon-gun (KR)

(21) Appl. No.: 19/362,523

(22) Filed: Oct. 20, 2025

(30) Foreign Application Priority Data
Jan. 15, 2025 (KR) 10-2025-0005919
Jun. 19, 2025 (KR) 10-2025-0081199

(51) Int. Cl. A61B 8/00; A61B 8/08; G16H 30/40

(52) U.S. Cl. CPC A61B 8/54; A61B 8/0858; A61B 8/463; A61B 8/5223; G16H 30/40

(57) ABSTRACT

Disclosed is an ultrasonic diagnostic apparatus including a probe configured to transmit an ultrasonic signal to an object and receive information about an echo signal reflected from the object, and a main body including a display, an input interface configured to receive user input, and a processor configured to obtain physical examination data about a sacrum skin lesion of the object from the input interface, obtain an ultrasonic image of the object based on the information about the echo signal obtained from the probe, detect a variation related to a sacral dimple on the ultrasonic image, classify the variation as any one of a normal variation and a pathological finding, and control the display to display information about the pathological finding on the ultrasonic image based on the variation being classified as the pathological finding.

### CROSS-REFERENCE TO RELATED APPLICATION(S)

This application is based on and claims priority under 35 U.S.C. §119 to Korean Patent Application Nos. 10-2025-0005919 and 10-2025-0081199, filed on January 15, 2025 and filed on June 19, 2025, in the Korean Intellectual Property Office, the disclosure of which is incorporated by reference herein in its entirety.

### BACKGROUND

### Field

The disclosure relates to an ultrasonic diagnostic apparatus and a control method thereof capable of assisting in the diagnosis of diseases related to an atypical sacral dimple.

### Description of the Related Art

Recently, in a medical field, various medical imaging apparatuses have been widely used to image and obtain information about biological tissues of a human body for the purpose of early diagnosis of various diseases or surgery. Representative examples of such medical imaging apparatuses may include ultrasonic imaging apparatuses, computed tomography (CT) apparatuses, and magnetic resonance imaging (MRI) apparatuses.

An ultrasonic imaging apparatus is a device that transmits an ultrasonic signal generated from a transducer of a probe to an object, and non-invasively obtains at least one image of a region inside the object (e.g., soft tissue or blood flow) by receiving information from the signal reflected from the object. An ultrasonic imaging apparatus may be used for medical purposes such as observing the inside of an object, detecting foreign substances, and measuring injury. Such an ultrasonic imaging apparatus is widely used together with other imaging apparatuses because the ultrasonic imaging apparatus has higher stability than an imaging apparatus using an X-ray, may display images in real time, and is safe because there is no radiation exposure.

In newborns and infants under three months of age, dimple (sacral dimple) examination using an ultrasonic diagnostic apparatus is possible because the spinal ossification has not yet fully progressed. Additionally, when an ultrasonic diagnostic apparatus is used, the ultrasonic diagnostic apparatus is more widely used because of being more accessible in terms of time and cost than when an MRI is used. However, there have been problems that lesion evaluation is performed differently depending on the skill and judgment of an ultrasound examiner, and in cases of spinal abnormalities, additional repetitive and cumbersome work is required to manually count and record vertebral levels.

### SUMMARY

It is an aspect of the disclosure to provide an ultrasonic diagnostic apparatus and a control method thereof capable of more quickly and accurately identifying an abnormal findings related to an atypical sacral dimple to easily assist in early treatment or preventive surgical decisions.

Additional aspects of the disclosure will be set forth in part in the description which follows and, in part, will be obvious from the description, or may be learned by practice of the disclosure.

An aspect of the disclosure provides a control method of an ultrasonic diagnostic apparatus including obtaining physical examination data about a sacrum skin lesion of an object from a user, obtaining an ultrasonic image of the object, detecting a variation related to a sacral dimple on the ultrasonic image, classifying the variation as any one of a normal variation and a pathological finding, and indicating information about the pathological finding on the ultrasonic image based on the variation being classified as the pathological finding.

Another aspect of the disclosure provides an ultrasonic diagnostic apparatus including a probe configured to transmit an ultrasonic signal to an object and receive information about an echo signal reflected from the object, and a main body including a display, an input interface configured to receive user input, and a processor configured to obtain physical examination data about a sacrum skin lesion of the object from the input interface, obtain an ultrasonic image of the object based on the information about the echo signal obtained from the probe, detect a variation related to a sacral dimple on the ultrasonic image, classify the variation as any one of a normal variation and a pathological finding, and control the display to display information about the pathological finding on the ultrasonic image based on the variation being classified as the pathological finding.

### BRIEF DESCRIPTION OF THE DRAWINGS

These and/or other aspects of the disclosure will become apparent and more readily appreciated from the following description of the embodiments, taken in conjunction with the accompanying drawings of which:

FIGS. 1 and 2 are block diagrams illustrating components of an ultrasonic imaging system according to an embodiment;

FIGS. 3 to 6 are views illustrating the ultrasonic imaging system according to an embodiment;

FIG. 7 is an example of a cross-sectional ultrasonic image of a spine according to an embodiment;

FIG. 8 is another example of a cross-sectional ultrasonic image of the spine according to an embodiment;

FIG. 9 is another example of a cross-sectional ultrasonic image of the spine according to an embodiment;

FIG. 10 illustrates an example in which a cross-sectional ultrasonic image of the spine includes a variant related to a sacral dimple according to an embodiment;

FIG. 11 illustrates another example in which a cross-sectional ultrasonic image of the spine includes a variant related to the sacral dimple according to an embodiment;

FIG. 12 illustrates another example in which a cross-sectional ultrasonic image of the spine includes a variant related to the sacral dimple according to an embodiment;

FIG. 13 illustrates another example in which a cross-sectional ultrasonic image of the spine includes a variant related to the sacral dimple according to an embodiment;

FIG. 14 illustrates an example in which a variation related to the sacral dimple included in a cross-sectional ultrasonic image of the spine according to an embodiment is classified as a pathological finding related to tethered cord syndrome;

FIG. 15 illustrates an example in which a variation related to the sacral dimple included in a cross-sectional ultrasonic image of the spine according to an embodiment is classified as a pathological finding related to tethered cord syndrome;

FIG. 16 is a view illustrating an example of a cross-sectional ultrasonic image of the spine on which predicted disease information related to the pathological finding, name information about the pathological finding, and anatomical position information about the pathological finding are displayed according to an embodiment;

FIG. 17 is a view illustrating an example of a cross-sectional ultrasonic image of the spine displayed with numerical value information about the pathological finding according to an embodiment;

FIG. 18 is a view illustrating another example of an ultrasonic image displayed with the anatomical position information about the pathological finding according to an embodiment;

FIG. 19 is a view illustrating another example of a cross-sectional ultrasonic image of the spine displayed with the anatomical position information about the pathological finding according to an embodiment;

FIG. 20 is a view illustrating another example of a cross-sectional ultrasonic image of the spine displayed with the anatomical position information about the pathological finding according to an embodiment; and

FIG. 21 is a control flowchart of an ultrasonic diagnostic apparatus according to an embodiment.

### DETAILED DESCRIPTION

This disclosure will explain the principles and disclose embodiments of the disclosure to clarify the scope of the claims of the disclosure and enable those skilled in the art to which the embodiments of the disclosure belong to practice the embodiments. The embodiments of the disclosure may be implemented in various forms.

Throughout this specification, like reference numbers refer to like components. This specification does not describe all components of the embodiments, and general contents in the technical field to which the disclosure belongs or overlapping contents between the embodiments will not be described. The "module" or "unit" used in the specification may be implemented as one or a combination of two or more of software, hardware, or firmware, and according to the embodiments, a plurality of "modules" or "units" may be implemented as a single element, or a single "module" or "unit" may include a plurality of elements.

The singular form of a noun corresponding to an item may include a single item or a plurality of items, unless the relevant context clearly indicates otherwise.

In this disclosure, each of phrases such as "A or B," "at least one of A and B," "at least one of A or B," "A, B or C," "at least one of A, B and C," and "at least one of A, B, or C" may include any one of the items listed together in the corresponding one of the phrases, or all possible combinations thereof.

The term "and/or" includes any combination of a plurality of related components or any one of a plurality of related components.

The terms such as "first," "second," "primary," and "secondary" may simply be used to distinguish a given component from other corresponding components, and do not limit the corresponding components in any other respect (e.g., importance or order).

The terms "front surface," "rear surface," "upper surface," "lower surface," "side surface," "left side," "right side," "upper portion," "lower portion," and the like used in the disclosure are defined with reference to the drawings, and the shape and position of each component are not limited by these terms.

The terms "comprises," "has," and the like are intended to indicate that there are features, numbers, steps, operations, components, parts, or combinations thereof described in the disclosure, and do not exclude the presence or addition of one or more other features, numbers, steps, operations, components, parts, or combinations thereof.

When any component is referred to as being "connected," "coupled," "supported," or "in contact" with another component, this includes a case in which the components are indirectly connected, coupled, supported, or in contact with each other through a third component as well as directly connected, coupled, supported, or in contact with each other.

When any component is referred to as being located "on" or "over" another component, this includes not only a case in which any component is in contact with another component but also a case in which another component is present between the two components.

Hereinafter, an ultrasonic apparatus according to various embodiments will be described in detail with reference to the accompanying drawings. When described with reference to the accompanying drawings, similar reference numbers may be assigned to identical or corresponding components and redundant description thereof may be omitted.

In this disclosure, an image may include a medical image obtained by a medical imaging apparatus such as a magnetic resonance imaging (MRI) apparatus, a computed tomography (CT) apparatus, an ultrasonic imaging apparatus, and an X-ray imaging apparatus.

In this disclosure, an ‘object’, which is subject to photography, may include a person, animal, or part thereof. For example, the object may include a part of a human body (an organ, etc.) or a phantom.

In this disclosure, an ‘ultrasonic image’ refers to an image of an object that has been generated or processed based on an ultrasonic signal transmitted to and reflected from the object.

Hereinafter, embodiments of the disclosure will be described in detail with reference to the drawings.

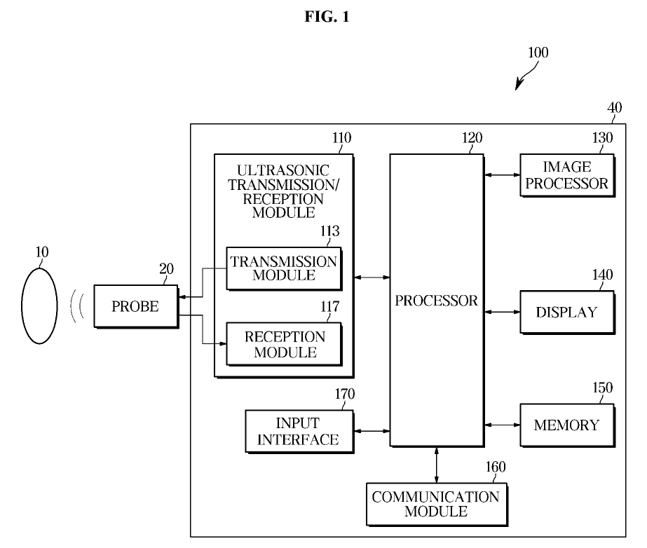

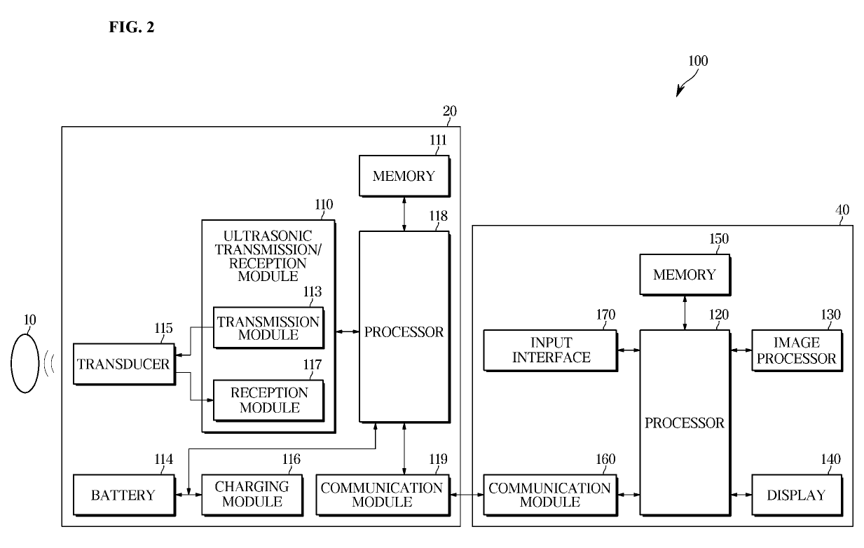

FIGS. 1 and 2 are block diagrams illustrating components of an ultrasonic imaging system according to an embodiment.

Referring to FIGS. 1 and 2, an ultrasonic imaging system 100 may include a probe 20 and an ultrasonic imaging apparatus 40.

The ultrasonic imaging apparatus 40 may be implemented not only in a cart type but also in a portable type. A portable ultrasonic imaging apparatus may include, for example, a smart phone, a laptop computer, a personal digital assistant (PDA), or a tablet PC, which includes a probe and an application, but is not limited thereto. The ultrasonic imaging apparatus 40 may also be implemented as an integrated probe.

The probe 20 may include a wired probe connected to the ultrasonic imaging apparatus 40 by wire to communicate with the ultrasonic imaging apparatus 40 by wire, a wireless probe wirelessly connected to the ultrasonic imaging apparatus 40 to communicate wirelessly with the ultrasonic imaging apparatus 40, and/or a hybrid probe connected to the ultrasonic imaging apparatus 40 by wire or wirelessly to communicate with the ultrasonic imaging apparatus 40 by wire or wirelessly.

According to various embodiments of the disclosure, as illustrated in FIG. 1, the ultrasonic imaging apparatus 40 may include an ultrasonic transmission/reception module 110, and as illustrated in FIG. 2, the probe 20 may include the ultrasonic transmission/reception module 110. According to various embodiments of the disclosure, both the ultrasonic imaging apparatus 40 and the probe 20 may also include the ultrasonic transmission/reception module 110.

According to various embodiments of the disclosure, the probe 20 may further include at least one or a combination of an image processor 130, a display 140, or an input interface 170. In the disclosure, a description of the ultrasonic transmission/reception module 110, the image processor 130, the display 140, or the input interface 170 included in the ultrasonic imaging apparatus 40 may also be applied to the ultrasonic transmission/reception module 110, the image processor 130, the display 140, or the input interface 170 included in the probe 20.

FIG. 1 is a control block diagram of the ultrasonic imaging system 100 in a case in which the probe 20 is a wired probe or a hybrid probe.

The probe 20 may include a plurality of transducers. The plurality of transducers may be implemented as a transducer array by being arranged in a predetermined arrangement. The transducer array may correspond to a one-dimensional (1D) array or a two-dimensional (2D) array. The plurality of transducers may transmit an ultrasonic signal to an object 10 in response to a transmission signal applied from a transmission module 113. The plurality of transducers may form a reception signal by receiving the ultrasonic signal (echo signal) reflected from the object 10. The probe 20 may be implemented as an integrated type with the ultrasonic imaging apparatus 40, or may be implemented as a separate type connected to the ultrasonic imaging apparatus 40 by wire. The ultrasonic imaging apparatus 40 may be connected to the one or more probes 20 depending on the implementation type.

In the case in which the probe 20 is a wired probe or a hybrid probe, the probe 20 may include a cable and a connector connectable to a connector of the ultrasonic imaging apparatus 40.

The probe 20 according to an embodiment may be implemented as a two-dimensional probe. In a case in which the probe 20 is implemented as a two-dimensional probe, the plurality of transducers included in the probe 20 may be arranged in two dimensions to form a two-dimensional transducer array.

For example, the two-dimensional transducer array may have a form in which a plurality of sub-arrays including the plurality of transducers arranged in a first direction is arranged in a second direction different from the first direction.

In the case in which the probe 20 according to an embodiment is implemented as a two-dimensional probe, the ultrasonic transmission/reception module 110 may include at least one of an analog beamformer or a digital beamformer. According to an embodiment, the two-dimensional probe may include at least one of the analog beamformer or the digital beamformer or a combination thereof depending on the implementation type.

A processor 120 controls the transmission module 113 to form a transmission signal to be applied to each of the transducers 115 in consideration of positions and focused points of the plurality of transducers 115 included in the probe 20.

The processor 120 may control a reception module 117 to generate ultrasonic data by converting reception signals received from the probe 20 into analog to digital and summing up the digitally converted reception signals in consideration of the positions and focused points of the plurality of transducers 115.

In the case in which the probe 20 is implemented as a two-dimensional probe, the processor 120 may calculate a time delay value for digital beamforming by each of the sub-arrays for each of the plurality of sub-arrays included in the two-dimensional transducer array. The processor 120 may also calculate a time delay value for analog beamforming for each of the transducers included in one of the plurality of sub-arrays. The processor 120 may control the analog beamformer and the digital beamformer to form a transmission signal to be applied to each of the plurality of transducers depending on the time delay values for analog beamforming and the time delay values for digital beamforming. The processor 120 may also control the analog beamformer to sum up the signals received from the plurality of transducers by each sub-array depending on the time delay values for analog beamforming. The processor 120 may also control the ultrasonic transmission/reception module 110 to convert the summed signals by each sub-array into analog to digital. The processor 120 may also control the digital beamformer to generate ultrasonic data by summing up the digitally converted signals depending on the time delay values for digital beamforming.

The image processor 130 generates or processes an ultrasonic image using the generated ultrasonic data.

The display 140 may display the generated ultrasonic image and a variety of information processed in the ultrasonic imaging apparatus 40 or the probe 20. The probe 20 or the ultrasonic imaging apparatus 40 may include the one or more displays 140 depending on the implementation type. The display 140 may also include a touch panel or a touch screen. The display 140 may also include a flexible display.

The processor 120 may control the overall operations of the ultrasonic imaging apparatus 40 and control operations of components of the ultrasonic imaging apparatus 40. The processor 120 may perform or control various operations or functions of the ultrasonic imaging apparatus 40 by executing programs or instructions stored in memory 150. The processor 120 may also control an operation of the ultrasonic imaging apparatus 40 by receiving a control signal from the input interface 170 or an external device.

The ultrasonic imaging apparatus 40 may include a communication module 160, and may be connected to and communicate with an external device (e.g., the probe 20, a server, a medical device, a portable device (a smart phone, tablet PC, wearable device, etc.)) through the communication module 160.

The communication module 160 may include one or more components enabling communication with an external device. The communication module 160 may include, for example, at least one of a short-range communication module, a wired communication module, or a wireless communication module.

The communication module 160 may also receive a control signal or data from the external device. The processor 120 may control the operation of the ultrasonic imaging apparatus 40 in response to the control signal received through the communication module 160. Also, the processor 120 may transmit a control signal to the external device through the communication module 160 to control the external device according to the transmitted control signal. The external device may operate in response to the control signal received from the ultrasonic imaging apparatus 40 or process data received from the ultrasonic imaging apparatus 40.

A program or application related to the ultrasonic imaging apparatus 40 may be installed on the external device. The program or application installed on the external device may control the ultrasonic imaging apparatus 40 or operate according to a control signal or data received from the ultrasonic imaging apparatus 40.

The external device may receive or download the program or application related to the ultrasonic imaging apparatus 40 from the ultrasonic imaging apparatus 40, the probe 20, or a server, and install and execute the program or application thereon. The ultrasonic imaging apparatus 40, the probe 20, or the server providing the program or application may include a recording medium storing instructions, commands, installation files, executable files, or related data of the program or application. The external device may also be sold with the program or application installed.

The memory 150 may store various data or programs for driving and controlling the ultrasonic imaging apparatus 40, inputted and outputted ultrasonic data, ultrasonic images, and the like.

The input interface 170 may receive user input for controlling the ultrasonic imaging apparatus 40. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

FIG. 2 illustrates a control block diagram of the ultrasonic imaging system 100 in a case in which the probe 20 is a wireless probe or a hybrid probe.

According to various embodiments of the disclosure, the ultrasonic imaging apparatus 40 illustrated in FIG. 2 may be replaced with the ultrasonic imaging apparatus 40 described with reference to FIG. 1.

According to various embodiments of the disclosure, the probe 20 described with reference to FIG. 1 may be replaced with the probe 20 to be described with reference to FIG. 2.

The probe 20 may include a display 112, the transmission module 113, a battery 114, the transducer 115, a charging module 116, the reception module 117, an input interface 109, a processor 118, and a communication module 119. FIG. 2 illustrates that the probe 20 includes both the transmission module 113 and the reception module 117, but the probe 20 may include only parts of components of the transmission module 113 and the reception module 117 depending on the implementation type, and the parts of the components of the transmission module 113 and the reception module 117 may be included in the ultrasonic imaging apparatus 40. According to an embodiment, the probe 20 may further include the image processor 130.

The transducer 115 may include a plurality of transducers. The plurality of transducers may be implemented as a transducer array by being arranged in a predetermined arrangement. The transducer array may correspond to a one-dimensional (1D) array or a two-dimensional (2D) array. The plurality of transducers may transmit an ultrasonic signal to the object 10 in response to a transmission signal applied from a transmission module 113. The plurality of transducers may form or generate an electrical reception signal by receiving the ultrasonic signal reflected from the object 10.

The charging module 116 may charge the battery 114. The charging module 116 may receive electric power from the outside. According to an embodiment, the charging module 116 may receive electric power wirelessly. According to an embodiment, the charging module 116 may also receive electric power by wire. The charging module 116 may transfer the received electric power to the battery 114.

The processor 118 controls the transmission module 113 to generate or form a transmission signal to be applied to each of the plurality of transducers in consideration of the positions and focused points of the plurality of transducers.

The processor 118 controls the reception module 117 to generate ultrasonic data by converting reception signals received from the transducers 115 into analog to digital and summing up the digitally converted reception signals in consideration of the positions and focused points of the plurality of transducers. According to an embodiment, in a case in which the probe 20 includes the image processor 130, the probe 20 may generate an ultrasonic image using the generated ultrasonic data.

In the case in which the probe 20 is implemented as a two-dimensional probe, the processor 118 may calculate the time delay value for digital beamforming by each sub-array for each of the plurality of sub-arrays included in the two-dimensional transducer array. The processor 118 may also calculate the time delay value for analog beamforming for each of the transducers included in one of the plurality of sub-arrays. The processor 118 may control the analog beamformer and the digital beamformer to form a transmission signal to be applied to each of the plurality of transducers depending on the time delay values for analog beamforming and the time delay values for digital beamforming. The processor 118 may also control the analog beamformer to sum up the signals received from the plurality of transducers by each sub-array depending on the time delay values for analog beamforming. The processor 118 may also control the ultrasonic transmission/reception module 110 to convert the summed signals by each sub-array into analog to digital. The processor 118 may also control the digital beamformer to generate ultrasonic data by summing up the digitally converted signals depending on the time delay values for digital beamforming.

The processor 118 may control the overall operations of the probe 20 and control operations of components of the probe 20. The processor 118 may perform or control various operations or functions of the probe 20 by executing programs or instructions stored in memory 111. The processor 118 may also control an operation of the probe 20 by receiving a control signal from the input interface 109 of the probe 20 or an external device (e.g., ultrasonic imaging apparatus 40). The processor 118 may also control the operation of the probe 20 by receiving a control signal from the input interface 109 or an external device. The input interface 109 may receive user input for controlling the probe 20. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

The display 112 may display an ultrasonic image generated by the probe 20, an ultrasonic image generated by processing ultrasonic data generated by the probe 20, an ultrasonic image received from the ultrasonic imaging apparatus 40, or a variety of information processed by the ultrasonic imaging system 100. The display 112 may further display state information about the probe 20. The status information about the probe 20 may include at least one of device information about the probe 20, battery status information about the probe 20, frequency band information about the probe 20, output information about the probe 20, information on whether the probe 20 is abnormal, setting information about the probe 20, or temperature information about the probe 20.

The probe 20 may include the one or more displays 112 depending on the implementation type. The display 112 may include a touch panel or touch screen. The display 112 may also include a flexible display.

The communication module 119 may wirelessly transmit the generated ultrasonic data or ultrasonic images to the ultrasonic imaging apparatus 40 through a wireless network. The communication module 119 may also receive a control signal and data from the ultrasonic imaging apparatus 40.

The ultrasonic imaging apparatus 40 may receive the ultrasonic data or ultrasonic images from the probe 20.

In an embodiment, the case in which the probe 20 includes the image processor 130 capable of generating ultrasonic images using the ultrasonic data, the probe 20 may transmit the ultrasonic data or the ultrasonic images generated by the image processor 130 to the ultrasonic imaging apparatus 40.

In an embodiment, a case in which the probe 20 does not include the image processor 130 capable of generating ultrasonic images using the ultrasonic data, the probe 20 may transmit the ultrasonic data to the ultrasonic imaging apparatus 40. The ultrasonic data may include ultrasonic raw data, and the ultrasonic images may refer to ultrasonic image data.

The ultrasonic imaging apparatus 40 may include the processor 120, the image processor 130, the display 140, the memory 150, the communication module 160, and the input interface 170.

The image processor 130 generates or processes ultrasonic images using the ultrasonic data received from the probe 20.

The display 140 may display the ultrasonic images received from the probe 20, ultrasonic images generated by processing the ultrasonic data received from the probe 20, or a variety of information processed in the ultrasonic imaging system 100. The ultrasonic imaging apparatus 40 may include the one or more displays 140 depending on the implementation type. The display 140 may also include a touch panel or a touch screen. The display 140 may also include a flexible display.

The processor 120 may control the overall operations of the ultrasonic imaging apparatus 40 and control the operations of the components of the ultrasonic imaging apparatus 40. The processor 120 may perform or control the various operations or functions of the ultrasonic imaging apparatus 40 by executing the programs or applications stored in the memory 150. The processor 120 may also control the operation of the ultrasonic imaging apparatus 40 by receiving a control signal from the input interface 170 or an external device.

The ultrasonic imaging apparatus 40 may include the communication module 160, and may be connected to and communicate with an external device (e.g., the probe 20, a server, a medical device, a portable device (a smart phone, tablet PC, wearable device, etc.)) through the communication module 160.

The communication module 160 may include one or more components enabling communication with the external device. The communication module 160 may include, for example, at least one of a short-range communication module, a wired communication module, or a wireless communication module.

The communication module 160 of the ultrasonic imaging apparatus 40 and the communication module 119 of the probe 20 may communicate using a network or a short-range wireless communication method. For example, the communication module 160 of the ultrasonic imaging apparatus 40 and the communication module 119 of the probe 20 may communicate using any one of wireless LAN, Wi-Fi, Bluetooth, ZigBee, Wi-Fi Direct (WFD), Infrared Data Association (IrDA), Bluetooth Low Energy (BLE), Near Field Communication (NFC), Wireless Broadband Internet (WiBro), World Interoperability for Microwave Access (WiMAX), Shared Wireless Access Protocol (SWAP), Wireless Gigabit Alliance (WiGig), RF communication, and a wireless data communication method including 60GHz millimeter wave (mm wave) short-range communication.

To this end, the communication module 160 of the ultrasonic imaging apparatus 40 and the communication module 119 of the probe 20 may include at least one of a wireless LAN communication module, a Wi-Fi communication module, a Bluetooth communication module, a ZigBee communication module, a Wi-Fi Direct (WFD) communication module, an Infrared Data Association (IrDA) communication module, a Bluetooth Low Energy (BLE) communication module, a Near Field Communication (NFC) module, a Wireless Broadband Internet (WiBro) communication module, a World Interoperability for Microwave Access (WiMAX) communication module, a Shared Wireless Access Protocol (SWAP) communication module, a Wireless Gigabit Alliance (WiGig) communication module, a RF communication module, or a 60GHz millimeter wave (mm wave) short-range communication module.

In an embodiment, the probe 20 may transmit device information (e.g., ID information) of the probe 20 using a first communication method (e.g., BLE) to the paired ultrasonic imaging apparatus 40, may be wirelessly paired with the ultrasonic imaging apparatus 40. The probe 20 may also transmit ultrasonic data and/or ultrasonic images to the paired ultrasonic imaging apparatus 40.

The device information about the probe 20 may include a variety of information related to a serial number, model name, battery state of the probe 20, or the like.

The ultrasonic imaging apparatus 40 may receive the device information (e.g., ID information) of the probe 20 from the probe 20 using the first communication method (e.g., BLE) and be wirelessly paired with the probe 20. The ultrasonic imaging apparatus 40 may also transmit an activation signal to the paired probe 20 and receive the ultrasonic data and/or ultrasonic images from the probe 20. In this case, the activation signal may include a signal for controlling the operation of the probe 20.

In an embodiment, the probe 20 may transmit the device information (e.g., ID information) of the probe 20 the ultrasonic imaging apparatus 40 using the first communication method (e.g., BLE) and be wirelessly paired with the ultrasonic imaging apparatus 40. The probe 20 may also transmit the ultrasonic data and/or ultrasonic images to the ultrasonic imaging apparatus 40 paired by the first communication method using a second communication method (e.g., 60 GHz millimeter wave, Wi-Fi).

The ultrasonic imaging apparatus 40 may receive the device information (e.g., ID information) of the probe 20 from the probe 20 using the first communication method (e.g., BLE) and be wirelessly paired with the probe 20. The ultrasonic imaging apparatus 40 may also transmit the activation signal to the paired probe 20 and receive the ultrasonic data and/or ultrasonic images from the probe 20 using the second communication method (e.g., 60 GHz millimeter wave, Wi-Fi).

According to an embodiment, the first communication method used to pair the probe 20 and the ultrasonic imaging apparatus 40 with each other may have a frequency band lower than a frequency band of the second communication method used by the probe 20 to transmit the ultrasonic data and/or ultrasonic images to the ultrasonic imaging apparatus 40.

The display 140 of the ultrasonic imaging apparatus 40 may display UIs (user interfaces) indicating the device information about the probe 20. For example, the display 140 may display UIs, which indicate identification information about the wireless ultrasonic probe 20, a pairing method indicating a pairing method with the probe 20, a data communication state between the probe 20 and the ultrasonic imaging apparatus 40, a method of performing data communication with the ultrasonic imaging apparatus 40, or the battery state of the probe 20.

In a case in which the probe 20 includes the display 112, the display 112 of the probe 20 may display the UIs indicating the device information about the probe 20. For example, the display 112 may display the UIs, which indicate the identification information about the wireless ultrasonic probe 20, the pairing method indicating the pairing method with the probe 20, the data communication state between the probe 20 and the ultrasonic imaging apparatus 40, the method of performing the data communication with the ultrasonic imaging apparatus 40, or the battery state of the probe 20.

The communication module 160 may receive a control signal or data from an external device. The processor 120 may control the operation of the ultrasonic imaging apparatus 40 in response to the control signal received through the communication module 160.

The processor 120 may also transmit a control signal to the external device through the communication module 160 to control the external device according to the transmitted control signal. The external device may operate according to the control signal received from the ultrasonic imaging apparatus 40 or process data received from the ultrasonic imaging apparatus 40.

The external device may receive or download the program or application related to the ultrasonic imaging apparatus 40 from the ultrasonic imaging apparatus 40, the probe 20, or a server, and install and execute the program or application thereon. The ultrasonic imaging apparatus 40, the probe 20, or the server providing the program or application may include a recording medium storing instructions, commands, installation files, executable files, or related data of the program or application. The external device may also be sold with the program or application installed.

The memory 150 may store various data or programs for driving and controlling the ultrasonic imaging apparatus 40, inputted and outputted ultrasonic data, ultrasonic images, and the like.

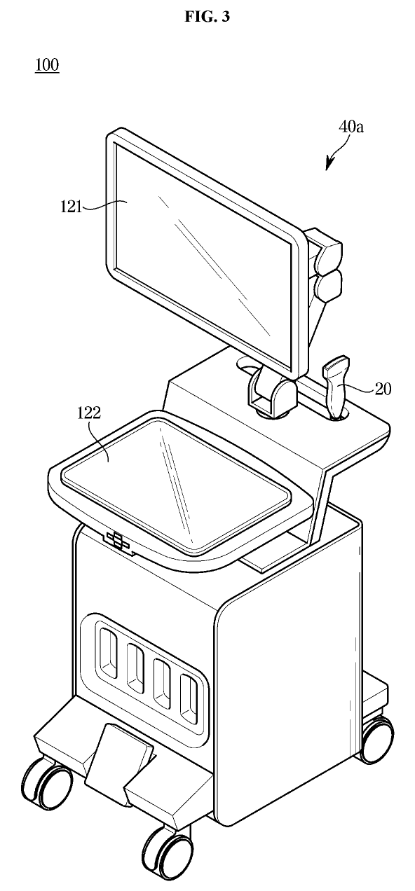

Examples of the ultrasonic imaging system 100 according to an embodiment of the disclosure will be described later with reference to FIGS. 3 to 6.

FIGS. 3 to 6 are views illustrating the ultrasonic imaging system according to an embodiment.

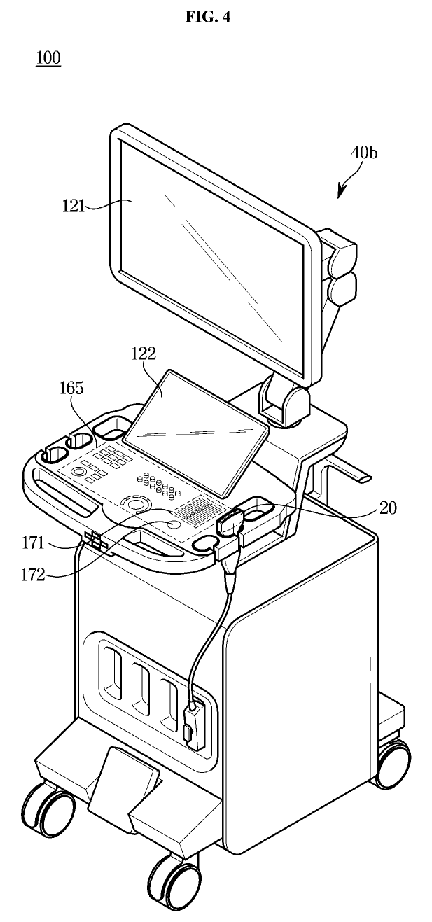

Referring to FIGS. 3 and 4, ultrasonic imaging apparatuses 40a and 40b may include a main display 121 and a sub display 122. The main display 121 and the sub display 122 may correspond to the display 140 of FIGS. 1 and 2. At least one of the main display 121 or the sub display 122 may be implemented as a touch screen. At least one of the main display 121 or the sub display 122 may display ultrasonic images or a variety of information processed in the ultrasonic imaging apparatuses 40a and 40b. Also, at least one of the main display 121 or the sub display 122 may be implemented as a touch screen, and receive input of data for controlling the ultrasonic imaging apparatuses 40a and 40b from a user by providing GUIs. For example, the main display 121 may display ultrasonic images, and the sub display 122 may display a control panel for controlling the display of the ultrasonic images in the form of GUIs. The sub display 122 may receive input of data for controlling the display of images through the control panel displayed in the form of GUIs. For example, a time gain compensation (TGC) button, a lateral gain compensation (LGC) button, a Freeze button, a trackball, a jog switch, a knob, or the like may be provided as GUIs on the sub display 122.

The ultrasonic imaging apparatuses 40a and 40b may control the display of ultrasonic images displayed on the main display 121 using the inputted control data. The ultrasonic imaging apparatuses 40a and 40b may be connected to the probe 20 by wire or wirelessly to transmit and receive ultrasonic signals to and from the object 10.

Referring to FIG. 4, the ultrasonic imaging apparatus 40b may further include a control panel 165 in addition to the main display 121 and the sub display 122. The control panel 165 may include a button, a trackball, a jog switch, a knob, and the like, and receive input of data for controlling the ultrasonic imaging apparatus 40b from the user. For example, the control panel 165 may include a TGC button 171, a Freeze button 172, and the like. The TGC button 171 is a button for setting a TGC value by each of depths of ultrasonic images. When input of the Freeze button 172 is detected while scanning an ultrasonic image, the ultrasonic imaging apparatus 40b may keep a state in which a frame image at that point in time is displayed, capture the frame image at that point in time, or store the frame image at that point in time.

The button, trackball, jog switch, knob, and the like included in the control panel 165 may be provided as GUIs on the main display 121 or the sub display 122. The ultrasonic imaging apparatuses 40a and 40b may be connected to the probe 20 to transmit and receive ultrasonic signals to and from the object 10.

The ultrasonic imaging apparatuses 40a and 40b may include various types of input/output interfaces such as speakers, LEDs, and vibration devices. For example, the ultrasonic imaging apparatuses 40a and 40b may output a variety of information in the form of graphics, sound, or vibration through the input/output interface. The ultrasonic imaging apparatuses 40a and 40b may also output various notifications or data through the input/output interface.

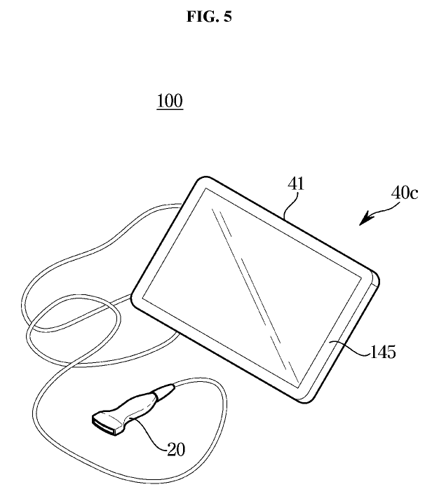

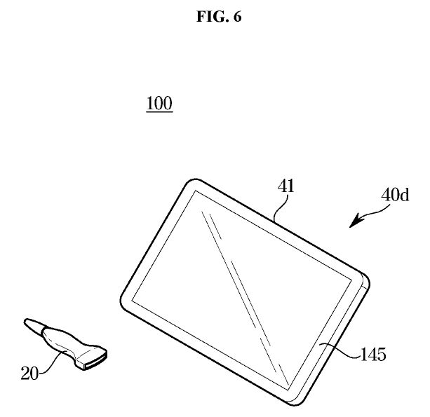

Referring to FIGS. 5 and 6, an ultrasonic imaging apparatuses 40c and 40d may be implemented in portable types. The portable ultrasonic imaging apparatuses 40c and 40d may include, for example, a smart phone, a laptop computer, a PDA, a tablet PC, and the like which includes a probe and an application, but are not limited thereto.

The ultrasonic imaging apparatus 40c may include a main body 41. Referring to FIG. 5, the probe 20 may be connected to one side of the main body 41 by wire. To this end, the main body 41 may include a connection terminal to and from which a cable connected to the probe 20 may be attached and detached. The probe 20 may include a cable including a connection terminal connectable to the main body 41.

Referring to FIG. 6, the probe 20 may be wirelessly connected to an ultrasonic imaging apparatus 40d. The main body 41 may include an input/output interface (e.g., a touch screen). Ultrasonic images, a variety of information processed in the ultrasonic imaging apparatus, or GUIs may be displayed on the input/output interface.

The ultrasonic imaging apparatus 40d and the probe 20 may establish communication or be paired using a short-range wireless communication. For example, the ultrasonic imaging apparatus 40d and the probe 20 may perform communication using Bluetooth, BLE, Wi-Fi, or Wi-Fi Direct.

The ultrasonic imaging apparatuses 40c and 40d may execute a program or application related to the probe 20 to control the probe 20 and output information related to the probe 20. The ultrasonic imaging apparatuses 40c and 40d may perform operations related to the probe 20 while communicating with a predetermined server. The probe 20 may be registered with the ultrasonic imaging apparatuses 40c and 40d or may be registered with the predetermined server. The ultrasonic imaging apparatuses 40c and 40d may communicate with the registered probe 20 and perform the operations related to the probe 20.

The ultrasonic imaging apparatuses 40c and 40d may also include various types of input/output interfaces such as speakers, LEDs, and vibration devices. For example, the ultrasonic imaging apparatuses 40c and 40d may output a variety of information in the form of graphics, sound, or vibration through the input/output interfaces. The ultrasonic imaging apparatuses 40c and 40d may also output various notifications or data through the input/output interfaces.

According to an embodiment of the disclosure, the ultrasonic imaging apparatus 40a, 40b, 40c, or 40d may process an ultrasonic image or obtain additional information from the ultrasonic image using an artificial intelligence (AI) model. According to an embodiment, the ultrasonic imaging apparatus 40a, 40b, 40c, or 40d may, using an AI model, generate an ultrasonic image, or perform processing of correction, image quality improvement, encoding, or decoding on an ultrasonic image. According to an embodiment of the disclosure, the ultrasonic imaging apparatus 40a, 40b, 40c, or 40d may also, using the AI model, perform processing of reference line definition, anatomical information obtainment, lesion information obtainment, surface extraction, boundary definition, length measurement, area measurement, volume measurement, or annotation creation, from an ultrasonic image.

The AI model may be provided on the ultrasonic imaging apparatus 40a, 40b, 40c, or 40d, or may be provided on the server.

The AI model may be implemented using various artificial neural network models or deep neural network models. In addition, the AI model may be learned and created using various machine learning algorithms or deep learning algorithms. The AI model may be implemented using, for example, a model such as a convolutional neural network (CNN), a recurrent neural network (RNN), a generative adversarial network (GAN), or a long short-term memory (LSTM).

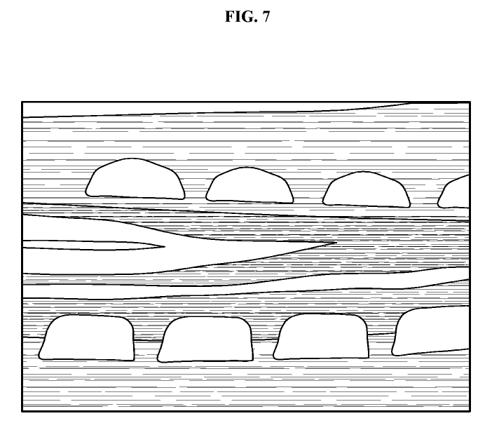

FIGS. 7 to 9 illustrate various examples of cross-sectional ultrasonic images of a spine according to an embodiment.

FIG. 7 is an example of a cross-sectional ultrasonic image of a spine according to an embodiment.

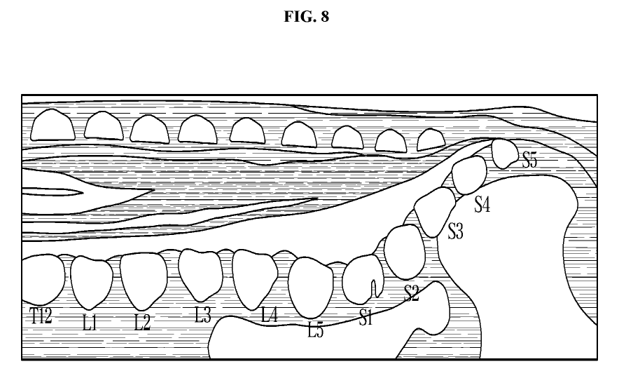

FIG. 8 is another example of a cross-sectional ultrasonic image of the spine according to an embodiment.

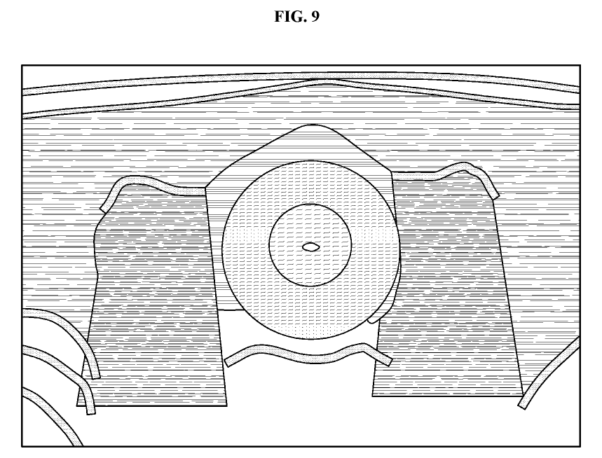

FIG. 9 is another example of a cross-sectional ultrasonic image of the spine according to an embodiment.

In order to determine whether there is an abnormality in a spine, it is necessary to obtain physical examination data inputted from a user (e.g., a doctor or sonographer) and cross-sectional ultrasonic images of the spine. This is because the accuracy of diagnosis of a neurological abnormality in a newborn may be improved through clinical features related to a sacral dimple that may be obtained from the physical examination data and imaging features related to the sacral dimple that may be obtained from the cross-sectional ultrasonic images of the spine. According to an embodiment, the physical examination data may correspond to all data obtained by the user through visual observation or physical examination (e.g., palpation, auscultation, etc.). The physical examination data may be referred to as clinical findings. For example, the physical examination data may include at least one of symptoms of a patient (e.g., pain, swelling, fever, etc.), physical examination results (e.g., palpable lumps, enlarged organs, skin condition, etc.), auscultation results (e.g., abnormal sounds heard in a heart, lungs, etc.), visual observations (e.g., discoloration, signs of trauma, deformities, etc.), or basic vital signs (e.g., a body temperature, blood pressure, pulse rate, respiration rate, etc.).

According to an embodiment, the physical examination data may include the clinical features related to the sacral dimple (hereinafter referred to as “clinical features”). That is, at least part of the physical examination data that are highly correlated with a typical or simple sacral dimple or an atypical sacral dimple may correspond to the clinical features.

The clinical features may include preset signs or symptoms related to the sacral dimple. For example, the clinical features may include at least one of a diameter of a sacral skin lesion (i.e., a skin lesion in a sacral area that may be visually observed by a user) observed in an object (e.g., a newborn), a distance from an anus to the sacral skin lesion, a positional relationship between the sacral skin lesion and a center line, a positional relationship between the sacral skin lesion and buttocks fold, or a skin abnormality.

According to an embodiment, the processor 120 of the ultrasonic imaging apparatus 40 may obtain the physical examination data. For example, the processor 120 may receive input of the physical examination data from the user through the input interface 170. As another example, the processor 120 may receive the physical examination data from an external device (e.g., a server device or other ultrasonic imaging apparatus) through the communication module 160.

The processor 120 may determine a sacral dimple type of the object based on the obtained physical examination data. In this case, the sacral dimple type may be determined as typical or atypical type. The typical sacral dimple may be referred to as a simple sacral dimple. Additionally, the sacral dimple type may be determined as one of type 1, type 2, and type 3. In this case, the typical sacral dimple may correspond to type 1. The atypical sacral dimple may include type 2 and type 3. In other words, the atypical sacral dimple may be classified as type 2 or type 3 depending on a degree of a pathological state thereof.

The processor 120 may determine the sacral dimple type of the object by comparing the clinical features included in the physical examination data with classification criteria pre-stored in the memory 150. In other words, the processor 120 may determine the sacral dimple type of the object based on a preset rule (or criterion)-based classification. For example, the classification criteria may include whether a diameter of a skin lesion is less than (or greater than) 5 mm, whether the skin lesion is located away from the center line, whether a distance that the skin lesion is spaced apart from the anus is less than (or greater than) 2.5 cm, or whether the skin lesion is accompanied by another skin lesion, such as hair. In this case, the processor 120 may update the pre-stored classification criteria (or threshold value of the classification criteria) through the communication module 160.

For example, the processor 120 may determine the sacral dimple type of the object as the typical sacral dimple or type 1 in a case in which the clinical features included in the physical examination data include the skin lesion diameter of less than 5 mm, the skin lesion located on the center line, and the distance of less than 2.5 cm from the anus to the skin lesion, and the skin lesion not accompanied by another skin lesion.

As another example, the processor 120 determine the sacral dimple type of the object as the atypical sacral dimple or type 2 in a case in which the clinical features included in the physical examination data include the skin lesion diameter of greater than 5 mm, the skin lesion located at an upper end of the center line, the distance of greater than 2.5 cm from the anus to the skin lesion, and a curvature of an end portion of the skin lesion fold.

As another example, the processor 120 determine the sacral dimple type of the object as the atypical sacral dimple or type 3 in a case in which the clinical features included in the physical examination data include the skin lesion diameter of greater than 5 mm, the skin lesion located away on the center line, the distance of greater than 2.5 cm from the anus to the skin lesion, and the skin lesion including other skin lesions (e.g., pigmentation, hemangioma, or hair, etc.).

According to various embodiments, the processor 120 may determine the sacral dimple type of the object by using the AI model based on the clinical features included in the physical examination data.

The processor 120 may learn the AI model using learning data. In this case, the learning data may include data labeled with correct values (typical/atypical or type 1/type 2/type 3) for the clinical features classified as the typical sacral dimple or the atypical sacral dimple, respectively.

For example, the processor 120 may extract the clinical features from the physical examination data and use the extracted clinical features as input data for the AI model. That is, the input data may include at least one of data about the diameter of the skin lesion, data about whether the skin lesion is spaced apart from the center line, data about the positional relationship between the skin lesion and the anus, or data about other skin lesions. The processor 120 may obtain the sacral dimple type as output data by inputting the input data into the learned AI model.

According to an embodiment, the processor 120 may detect a variation on an ultrasonic image. The variation may include any differences appearing on the ultrasonic image. For example, the variation may include a tumor, cyst, inflammation, abnormal reflection (echo) pattern, or difference in the shape of an organ or tissue.

According to an embodiment, the processor 120 may adjust an identification sensitivity based on the determined sacral dimple type in detecting the variations. The adjusting of the identification sensitivity may include adjusting at least one factor that determines how sensitively a variation is detected on the ultrasonic image.

For example, the processor 120 may decrease the identification sensitivity when the sacral dimple type is determined to be a typical sacral dimple. The decreasing of the identification sensitivity may include detecting only relatively large and distinct abnormal symptoms and ignoring small variations.

For example, the processor 120 may increase the identification sensitivity when the sacral dimple type is determined to be an atypical sacral dimple. The increasing of the identification sensitivity may include monitoring even small or subtle variations.

For example, the processor 120 may adjust the identification sensitivity depending on the degree of the pathological state thereof when the sacral dimple type is determined to be an atypical sacral dimple. For example, the processor 120 may increase the identification sensitivity more when the sacral dimple type is determined to be type 3 than when the sacral dimple type is determined to be type 2.

For example, the adjusting of the identification sensitivity may include adjusting an ultrasonic signal intensity and/or resolution. For example, the processor 120 may keep a preset default ultrasonic signal intensity and/or basic resolution when the sacral dimple type is determined to be the typical sacral dimple. On the other hand, the processor 120 may amplify the ultrasonic signal to improve a signal-to-noise ratio (SNR) or adjust a frequency of the ultrasonic signal to increase the resolution when the sacral dimple type is determined to be the atypical sacral dimple.

For example, the adjusting of the identification sensitivity may include adjusting an image contrast enhancement factor (CEF). The contrast enhancement factor (CEF) may be a value for determining how much a contrast between a variation and normal tissue is enhanced in an ultrasonic image. For example, the processor 120 may keep an image contrast enhancement index at a standard value when the dimple type is determined to be the typical sacral dimple. On the other hand, the processor 120 may increase the image contrast enhancement index when the dimple type is determined to be the atypical sacral dimple. Accordingly, the contrast is enhanced so that variations may be clearly displayed on the ultrasonic image.

In various examples, the processor 120 may adjust the identification sensitivity by adjusting various factors in addition to the factors described above. Any method that may be easily adopted by a person skilled in the art as a method for adjusting the identification sensitivity may be adopted as an element adjusted depending on the sacral dimple type in the disclosure. According to an embodiment, the processor 120 may analyze the obtained ultrasonic image based on the determined identification sensitivity.

According to an embodiment, the processor 120 may obtain various types of cross-sectional ultrasonic images of the spine depending on arrangement directions of the object 10 and the probe 20. For example, the processor 120 may obtain at least one of a long-axis spine cross-section ultrasonic image (longitudinal image) or a short-axis spine cross-section ultrasonic image (transverse image).

FIG. 7 illustrates a long-axis spine cross-sectional ultrasonic image (longitudinal image), which is an example of a cross-sectional ultrasonic image of the spine according to an embodiment. The long-axis spine cross-sectional ultrasonic image may correspond to an ultrasonic image obtained by disposing the probe 20 parallel to an axis of the spine (a direction in which vertebral bodies and intervertebral discs are arranged). The long-axis spine cross-sectional ultrasonic image is advantageous for observing a continuous structure of the spine at length.

FIG. 8 illustrates a panoramic ultrasonic image, which is an example of a long-axis spine cross-sectional ultrasonic image according to an embodiment. The long-axis spine cross-sectional ultrasonic image may correspond to a panoramic ultrasonic image. The panoramic ultrasonic image may correspond to a long-axis spine cross-sectional ultrasonic image generated by synthesizing multiple frames obtained while the user slowly moves the probe 20 along a spinal axis. According to various embodiments, the long-axis spine cross-sectional ultrasonic image may correspond to a dual ultrasonic image. The dual ultrasonic image may correspond to an ultrasonic image that shows two different image modes or points in time simultaneously.

According to an embodiment, the processor 120 may recognize an anatomical position of the spine and display an indicator indicating the position along with the cross-sectional ultrasonic image of the spine. The spine may include a cervical spine (C), thoracic spine (T), lumbar spine (L), sacrum (S), and coccyx (Co). The cervical spine is a neck portion and may include C1 to C7 (seven bones). The thoracic spine is a thoracic (chest) portion and may include T1 to T12 (twelve bones). For example, T1 is a first thoracic spine and may correspond to a portion connected to the cervical spine. The lumbar spine is a waist portion and may include L1 to L5 (five bones). L1 is a first lumbar spine and L5 is a last lumbar spine, which may correspond to the portions connected to the sacrum. The sacrum is a pelvic portion and may include S1 to S5 (five bones fused to form one sacrum). S1 may correspond to a first segment of the sacrum, and S2 to S3 may correspond to a central portion of the sacrum. The coccyx may correspond to a tailbone. The recognizing of the anatomical position of the spine may include recognizing the order of the aforementioned components configuring the spine.

According to one embodiment, the processor 120 may identify a lumbosacral junction (L5/S1 positions). The lumbosacral junction is a region in which the lumbar spine and sacrum of the spine meet, may refer to generally a position between L5 and S1. The lumbosacral junction may be an index region for identifying L1 to L5 and S1 to S5.

An acute angle may be formed between the lumbar spine and the sacrum at the lumbosacral junction. According to an embodiment, the processor 120 may recognize the lumbosacral junction based on a position of the acute angle when the acute angle is recognized.

A distinct acute angle at the lumbosacral junction may not be observed on a single ultrasonic image. Accordingly, the processor 120 may obtain a dual ultrasonic image or a panoramic ultrasonic image, and identify the continuous structure of the spine based on the dual ultrasonic image or the panoramic ultrasonic image. Through this, the processor 120 may calculate or recognize the order of anatomical structures along the spinal axis. Through this, the processor 120 may recognize the lumbosacral junction.

Thereafter, the processor 120 may display indicators on the ultrasonic image to indicate the anatomical structures along the spinal axis. For example, as illustrated in FIG. 8, an indicator (T12) indicating a portion of the thoracic spine connected to the lumbar spine (e.g., lumbar spine 1 (L1)) may be displayed on an ultrasonic image. As another example, an indicator (L5) indicating a portion connected to the sacrum as the last lumbar spine may be displayed on the ultrasonic image.

FIG. 9 illustrates a short-axis spine cross-sectional ultrasonic image (transverse image), which is another example of a spine cross-sectional ultrasonic image according to an embodiment. The short-axis spine cross-sectional ultrasonic image is a cross-sectional image obtained by disposing the ultrasonic probe 20 perpendicular to the axis of the spine, and through this image, transverse section structures of the spine, such as a spinal canal, nerve roots, a vertebral body transverse section, surrounding muscles, and blood vessels may be observed.

According to an embodiment, when a variation is detected, the processor 120 may classify the detected variation as any one of a normal variation and a pathological finding. The normal variation is a change that may differ from person to person anatomically or physiologically, but may correspond to a difference appearing within a normal range. That is, when the detected variation is classified as a normal variation, it is not a disease or abnormal state, and therefore treatment may not be necessary. On the other hand, the pathological finding may correspond to a case in which the variation detected on the ultrasonic image is outside the normal range. That is, when the detected variation is classified as a pathological finding, it may be clinically significant and may require additional examination or medical intervention. To be clinically significant may include being an objective index or basis for a diagnosis of a disease.

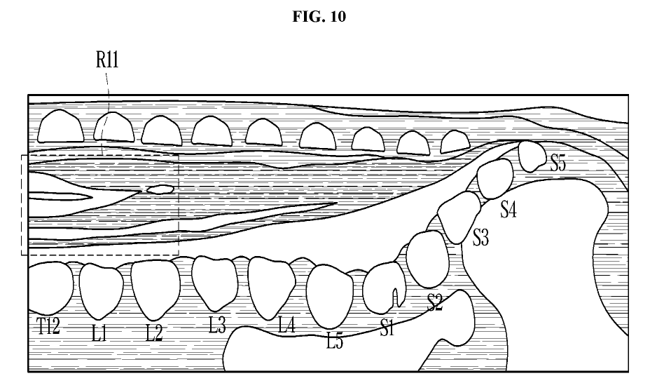

The processor 120 may classify the variation detected on the ultrasonic image as a pathological finding based on the anatomical criteria. In this case, the anatomical criteria for classifying a variation may be pre-stored in the memory 150. Additionally, according to various embodiments, the anatomical criteria for classifying variations may be changed based on user input received from the input interface 170. Hereinafter, detailed descriptions will be provided with reference to FIGS. 10 to 15.

According to an embodiment, the processor 120 may detect a variation related to the sacral dimple in the cross-sectional ultrasonic image of the spine. The processor 120 may determine whether a variation related to the sacral dimple is detected in each of a plurality of the cross-sectional ultrasonic images of the spine.

At this time, the processor 120 may perform contrast and brightness adjustment for clarifying an edge of the variation in a preprocessing process before detecting the variation. Additionally, the processor 120 may remove noise or adjust an echo intensity in order to improve the quality of the cross-sectional ultrasonic image of the spine in the preprocessing process. Accordingly, the processor 120 may determine an edge or anatomical position of the variation related to the sacral dimple.

According to various embodiments, the processor 120 may use a machine learning model in order to identify a variation related to the sacral dimple or a size, shape, or position of the variation. For example, the processor 120 may distinguish between a variation related to the sacral dimple and a surrounding structure surrounding thereof using a deep learning-based image segmentation algorithm. The processor 120 may also perform edge detection and texture analysis using the machine learning model. The processor 120 may also automatically detect a variation related to the sacral dimple through the artificial intelligence model that has learned past data and the labeled ultrasonic image (i.e., learning data).

FIG. 10 illustrates an example in which a cross-sectional ultrasonic image of the spine includes a variant related to a sacral dimple according to an embodiment.

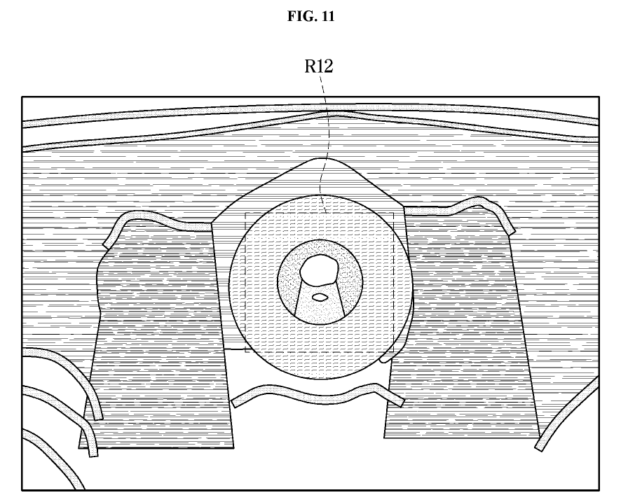

FIG. 11 illustrates another example in which a cross-sectional ultrasonic image of the spine includes a variant related to the sacral dimple according to an embodiment.

According to FIG. 10, the long-axis spine cross-sectional ultrasonic image may include a spinal cord filar cyst as a variation related to the sacral dimple. Also, according to FIG. 11, the short-axis spine cross-sectional ultrasonic image may include the spinal cord filar cyst as a variation related to the sacral dimple.

The filum terminale corresponds to a structure of being connected from the conus medullaris to the sacrum. When a cyst develops in the filum terminale, the cyst may appear as a thin-walled fusiform structure positioned within the filum terminale on the cross-sectional ultrasonic image of the spine. The processor 120 may determine whether the spinal cord filar cyst is present in the cross-sectional ultrasonic image of the spine.

For example, referring to FIG. 10, it may be checked that the spinal cord filar cyst is observed near L2 in the long-axis spine cross-section ultrasonic image in R11. Accordingly, the processor 120 may determine that a variation is detected in the cross-sectional ultrasonic image of the spine.

As another example, referring to FIG. 11, it may be checked that the filar cyst in the short-axis spine cross-sectional ultrasonic image in R12 is observed as a structure of being positioned within the filum terminale and surrounded by a thin wall at the center. Accordingly, the processor 120 may determine that the spinal cord filar cyst is detected by distinguishing from a dilatation of a central canal in the cross-sectional ultrasonic image of the spine.

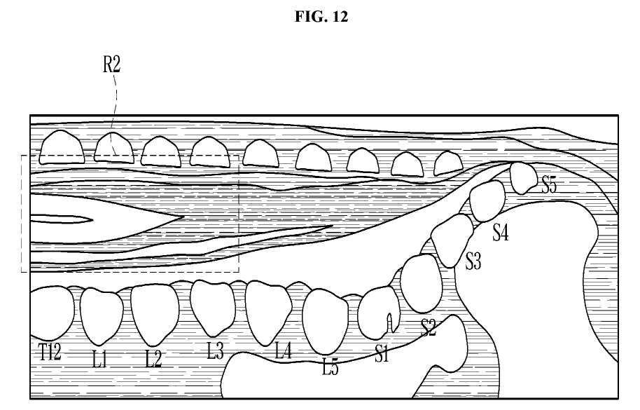

FIG. 12 illustrates another example in which a cross-sectional ultrasonic image of the spine includes a variant related to the sacral dimple according to an embodiment.

According to FIG. 12, the long-axis spine cross-sectional ultrasonic image may include a lower conus medullaris as a variation related to the sacral dimple.

The lower conus medullaris corresponds to an end of the conus medullaris being positioned as a normal position, that is, not being located near lumbar spine 1 (L1) and lumbar spine 2 (L2), but being located further down (toward the tailbone). The processor 120 may determine whether the lower conus medullaris is included in the cross-sectional ultrasonic image of the spine.

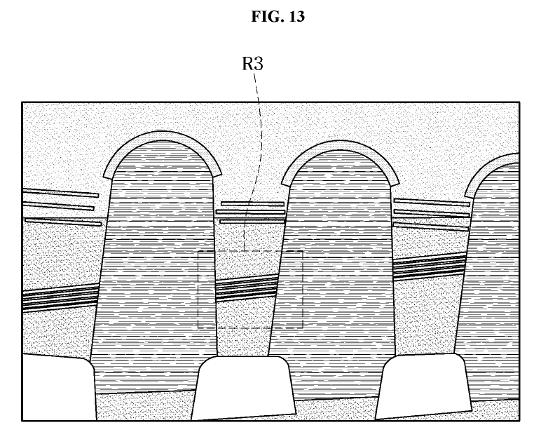

FIG. 13 illustrates another example in which a cross-sectional ultrasonic image of the spine includes a variant related to an atypical sacral dimple according to an embodiment.

According to FIG. 13, the long-axis spine cross-sectional ultrasonic image may include a thickened filum terminale(also referred to as “thickened spinal cord filum terminale”) as a variation related to the sacral dimple. The thickened filum terminale corresponds to a state in which the filum terminale originating from the conus medullaris of the spinal cord is abnormally thickened. The thickened filum terminale may be observed as hyperechoic on an ultrasonic image. The processor 120 may determine whether the thickened filum terminale is included in the cross-sectional ultrasonic image of the spine.

For example, referring to FIG. 13, it may be checked that the filum terminale thickened in R3 appears with a clear echo difference from the surrounding tissue. Accordingly, the processor 120 may determine that the cross-sectional ultrasonic image of the spine includes the thickened filum terminale.

According to various embodiments, although not illustrated in the drawings, the cross-sectional ultrasonic image of the spine may include variations related to the sacral dimple, such as defects in a lower portion of the spine, fatty tissue in a sacrum region, asymmetry of neural tissue, or immature neural canal closure.

According to various embodiments, the processor 120 may determine that a plurality of the variations is included in the cross-sectional ultrasonic image of the spine.

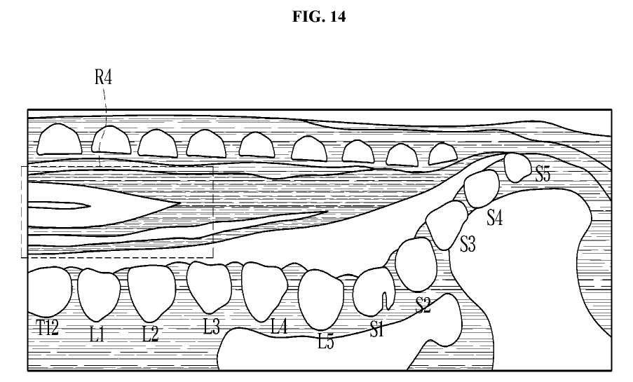

FIG. 14 illustrates an example in which a variation related to the sacral dimple included in a cross-sectional ultrasonic image of the spine according to an embodiment is classified as a pathological finding related to tethered cord syndrome.

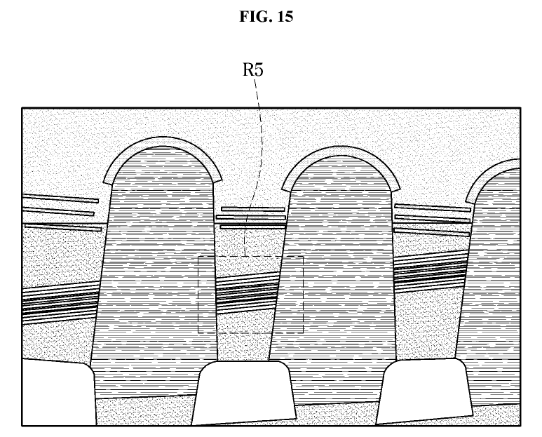

FIG. 15 illustrates an example in which a variation related to the sacral dimple included in a cross-sectional ultrasonic image of the spine according to an embodiment is classified as a pathological finding related to tethered cord syndrome.

The processor 120 may classify a variation related to the sacral dimple as any one of a normal variation and a pathological finding based on a preset anatomical criterion when detecting the variation related to the sacral dimple in the cross-sectional ultrasonic image of the spine.

According to FIG. 14, the processor 120 may classify a variation related to the sacral dimple as a pathological finding related to the tethered cord syndrome.

The processor 120 may classify the lower conus medullaris as any one of a normal variation and a pathological finding based on a position of the conus medullaris when the lower conus medullaris is detected. An anatomical criterion for classifying the lower conus medullaris as any one of a normal variation and a pathological finding may correspond to whether the end of the conus medullaris is positioned below lumbar spine 2 (L2).

For example, the processor 120 may classify the conus medullaris as the normal variation when the conus medullaris is positioned near lumbar spine 2 (L2) and is not lowered below lumbar spine 2 (L2). In this case, the lower conus medullaris classified as the normal variation may be referred to as a borderline lower conus medullaris.

The conus medullaris may exert a force of pulling downward on the spinal cord when the filum terminale does not regress in a fetal development process and remains in a thick state. Accordingly, the conus medullaris may be positioned lower (below L2-L3) than the normal position (near L1-L2). The processor 120 may classify the conus medullaris as a pathological finding when the conus medullaris is positioned lower (below L2-L3) than the normal position (near L1-L2).

For example, referring to FIG. 14, it may be checked that the end of the conus medullaris in R4 is positioned near L2-L3. Accordingly, the processor 120 may classify the lower conus medullaris, which is a variation included in the cross-sectional ultrasonic image of the spine, as a pathological finding.

According to various embodiments, the anatomical criterion for classifying a variation as any one of a normal variation and a pathological finding may be changed based on the user input received from the input interface 170. For example, when a newborn is an object, the user may input the user input to change the anatomical criterion for classifying the lower conus medullaris as any one of a normal variation and a pathological finding to whether the end of the conus medullaris is positioned lower than a vicinity (L2-L3) between lumbar spine 2 and a lumbar spine 3, through the input interface 170. Accordingly, the processor 120 may classify the lower conus medullaris as a normal variation when the end of the conus medullaris is positioned near L2-L3 based on the user input received. Hereinafter, only whether the end of the conus medullaris is positioned below lumbar spine 2 (L2) will be exemplarily explained as an example, as the anatomical criterion for classifying the lower conus medullaris as any one of a normal variation and a pathological finding, but this is only one example and the disclosure should not be construed as being limited thereto.

In this case, a lower spinal cord classified as the pathological finding may be related to the tethered cord syndrome. In other words, when the lower spinal cord classified as the pathological finding is included in the ultrasonic image, the user may diagnose the tethered cord syndrome.

However, the user does not necessarily diagnose the tethered cord syndrome just because the lower spinal cord is detected as a pathological finding on the ultrasonic image, and will be able to ultimately diagnose the tethered cord syndrome based on whether another pathological finding is also detected, such as thickened filum terminale, reduced or absent spinal cord movement/pulsation, or spinal cord lipomas.

As an example, although not illustrated in FIG. 14, in the case of tethered cord syndrome, the filar cyst may be accompanied by the thickened filum terminale with the lower conus medullaris as a pathological finding. When only the filar cyst is detected on an ultrasonic image, the user may not diagnose the tethered cord syndrome. On the other hand, when the filar cyst is detected together with the thickened filum terminale as a pathological finding along with the lower conus medullaris as a pathological finding on the ultrasonic image, the user may diagnose the tethered cord syndrome.

That is, only when detected along with the lower conus medullaris and the thickened filum terminale as pathological findings, the filar cyst may correspond to a pathological finding related to the tethered cord syndrome.

In other words, the filar cyst detected as a variation may be classified as the pathological finding. In this case, an anatomical criterion for classifying the filar cyst as any one of a normal variation and a pathological state may correspond to whether another pathological finding is detected on the ultrasonic image.

According to FIG. 15, the processor 120 may classify a variation related to the sacral dimple as a pathological finding related to the tethered cord syndrome.

When a thickened spinal cord filum terminale is detected, the processor 120 may classify the thickened spinal cord filum terminale as any one of a normal variation and a pathological finding based on a thickness of the filum terminale.

For example, processor 120 may classify the filum terminale as the normal variation when the thickness of the filum terminale is measured to be less than 2 mm. In this case, the thickened spinal cord filum terminale classified as the normal variation may be referred to as a borderline thickened filum terminale.

The processor 120 may classify as a pathological finding when the thickness of the filum terminale exceeds 2 mm. In this case, the thickened spinal cord filum terminale classified as the pathological finding may be related to the tethered cord syndrome. In other words, when the thickened spinal cord filum terminale classified as the pathological finding is included in the ultrasonic image, the user may diagnose the tethered cord syndrome. However, the tethered cord syndrome is not necessarily diagnosed only because the thickened spinal cord filum terminale is detected as a pathological finding on an ultrasonic image, the user will be able to ultimately the diagnose tethered cord syndrome as a pathological finding based on whether another pathological finding, such as a lower spinal cord, is also detected.

That is, an anatomical criterion for classifying the thickened spinal cord filum terminale as anyone of a normal variation and a pathological condition may correspond to whether the thickness of the thickened filum terminale exceeds 2 mm.

For example, the thickness of the thickened filum terminale shown in R5 of FIG. 15 may exceed 2 mm. Accordingly, the processor 120 may classify the thickened spinal cord filum terminale included in the cross-sectional ultrasonic image of the spine as a pathological finding.

According to various embodiments, the processor 120 may classify a variation related to the sacral dimple as a pathological finding related to a neurological disorder other than the tethered cord syndrome.

For example, although not shown in the drawing, the processor 120 may classify a variation related to the sacral dimple as a pathological finding related to spina bifida, caudal regression syndrome, spinal lipoma, meningocele, or central canal dilatation. In this case, the anatomical criteria for classifying the detected variation as any one of a normal variation and a pathological finding related to the spina bifida, caudal regression syndrome, spinal lipoma, meningocele, or central canal dilatation may be stored in the memory 150.

According to an embodiment, when the variation is classified as the pathological finding, the processor 120 may indicate information about the pathological finding on an ultrasonic image. Accordingly, the processor 120 may assist medical staff in making accurate diagnoses and improve diagnostic reliability of a patient.

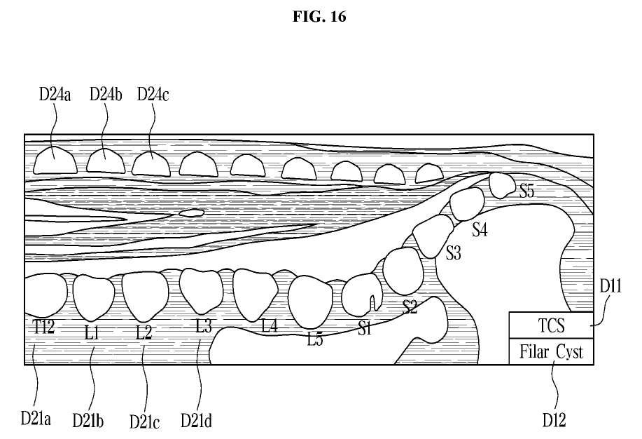

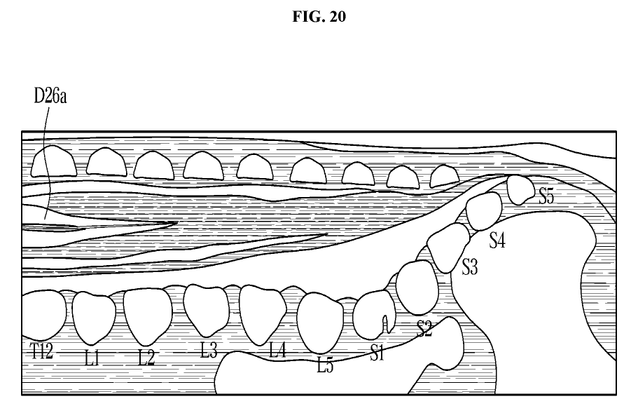

Specific descriptions will be provided below with reference to FIGS. 16 and 20. According to an embodiment, the processor 120 may control the display 140 to output information about a variation detected on an ultrasonic image.

The information about the variation may include information about a normal variation.

According to an embodiment, when a variation included in an ultrasonic image is classified as the normal variation, the processor 120 may display information about the normal variation on the ultrasonic image. For example, the information about the normal variation may include at least one of name information about the normal variation, anatomical position information about the normal variation, or numerical information about the normal variation. According to an embodiment, the processor 120 may display at least one of the name information, anatomical position information, or additional information about the normal variation using a graphic indicator or a text indicator.

For example, the processor 120 may display information about the lower conus medullaris as the lower conus medullaris, which is a variation detected on the ultrasonic image, is classified as the normal variation (e.g., when an end of the lower conus medullaris is positioned near lumbar spine 2 (L2)). The displaying of the information about the lower conus medullaris as a normal variation may include displaying at least one of name information, anatomical position information, or numerical information about the lower conus medullaris as a normal variation. The processor 120 may display the information about the lower conus medullaris as a normal variation using the graphical indicator or the text indicator. The processor 120 may display the information about the lower conus medullaris as a normal variation at a preset position with a fixed value.

According to an embodiment, the information about the variation may include information about a pathological finding. For example, the information about the pathological finding may include at least one of name information about the pathological finding, predicted disease information related to the pathological finding, anatomical position information about the pathological finding, or numerical information about the pathological finding.

According to an embodiment, the processor 120 may display name information about a pathological finding on an ultrasonic image when a variation included in the ultrasonic image is classified as the pathological finding. When there is a plurality of ultrasonic images, the processor 120 may display names of pathological findings included in the ultrasonic images, respectively.

For example, when the thickened filum terminale detected on an ultrasonic image is classified as the pathological finding, the processor 120 may indicate the thickened filum terminale, which is a name of the pathological finding, on the ultrasonic image.

As another example, when the filar cyst detected on an ultrasonic image is classified as the pathological finding, the processor 120 may display the filar cyst, which is a name of the pathological finding, on the ultrasonic image.

According to an embodiment, the processor 120 may display the predicted disease information about a pathological finding on an ultrasonic image when a variation included in the ultrasonic image is classified as the pathological finding. When there is a plurality of ultrasonic images, the processor 120 may display the predicted disease information about the pathological finding in each of the ultrasonic images. The predicted disease information may include name information about a predicted disease related to the pathological finding.

For example, when the thickened filum terminale detected on an ultrasonic image is classified as the pathological finding, the processor 120 may display the tethered cord syndrome, which is a predicted disease related to the thickened filum terminale, on the ultrasonic image.

As another example, when the filar cyst detected on an ultrasonic image is classified as the pathological finding, the processor 120 may display the tethered cord syndrome, which is a predicted disease related to the filar cyst, on an ultrasonic image.

Data about the name of each pathological finding and/or predicted disease information related to each pathological finding may be stored in the memory 150. Additionally, the ultrasonic diagnostic apparatus 40 may add and change data about the name of each pathological finding and/or predicted disease information related to each pathological finding by communicating with an external device (e.g., a server device) through the communication module 160.

According to an embodiment, the processor 120 may display the name information about the pathological finding and/or predicted disease information related to the pathological finding using the graphic indicator or the text indicator.

FIG. 16 is a view illustrating an example of a cross-sectional ultrasonic image of the spine on which predicted disease information related to the pathological finding, name information about the pathological finding, and anatomical position information about the pathological finding are displayed according to an embodiment.

According to an embodiment, the processor 120 may display at least one predicted disease information related to a predetermined pathological finding on an ultrasonic image. The at least one predicted disease information to be displayed on the ultrasonic image in relation to the predetermined pathological finding may be preset and stored in the memory 150. According to various embodiments, the processor 120 may receive the at least one predicted disease information from an external device (e.g., a server device) through the communication module 160.

The processor 120 may display preset predicted disease information as a fixed value at a preset position when the variation detected on the ultrasonic image is classified as the predetermined pathological finding. In this case, the preset predicted disease information may correspond to a predicted disease most frequently diagnosed when the pathological finding is detected. According to various embodiments, the processor 120 may change and/or add to the preset predicted disease information based on the user input received through the input interface 170.

For example, as the filar cyst is detected on the ultrasonic image along with the lower conus medullaris and thickened filum terminale as pathological findings, the processor 120 may display the tethered cord syndrome (TCS), which is preset predicted disease information related to the filar cyst, spinal cord lipoma, and lower spinal cord spondylosis, on the ultrasonic image (D11). As illustrated in FIG. 16, the processor 120 may classify the detected lower spinal cord as a lower spinal cord as a pathological finding, as the conus medullaris is positioned between L2 and L3. Additionally, although not shown in FIG. 16, the processor 120 may classify the detected thickened filum terminale as a thickened filum terminale as a pathological finding as the thickness of the thickened filum terminale exceeds 2 mm in the short-axis ultrasonic image (e.g., FIG. 15). Additionally, as illustrated in FIG. 16, the processor 120 may classify the detected filar cyst as a filar cyst as a pathological finding as the filar cyst is detected along with the lower spinal cord and thickened filum terminale as pathological findings.

In this case, with regard to the lower spinal cord, thickened filum terminale and, filar cyst as pathological findings, which are classified as pathological findings, the tethered cord syndrome may be preset as one of multiple predicted disease information to be displayed on the ultrasonic image and stored in memory 150. Accordingly, when the lower spinal cord, thickened filum terminale, and filar cyst detected on the ultrasonic image are classified as pathological findings, the processor 120 may display the tethered cord syndrome which is the preset predicted disease information as a fixed value, at the preset position (e.g., D11).

According to an embodiment, the processor 120 may display name information about the predetermined pathological finding on an ultrasonic image. The processor 120 may display the name information about the pathological finding on an ultrasonic image when a variation detected on the ultrasonic image is classified as the predetermined pathological finding. According to an embodiment, when a plurality of variations is detected and the plurality of variations is classified as pathological findings, the processor 120 may display name information about the highest priority pathological finding on the ultrasonic image according to a preset priority. In this case, the processor 120 may set and/or change the preset priority based on the user input received through the input interface 170.

For example, the processor 120 may display name information about the filar cyst as a pathological finding (D12) as the filar cyst is detected on the ultrasonic image along with the lower conus medullaris and thickened filum terminale as pathological findings. When the filar cyst detected on an ultrasonic image is classified as the pathological finding, the processor 120 may display the name information about the filar cyst as a pathological finding as a fixed value at a preset position (e.g., D12).

According to an embodiment, the processor 120 may display the anatomical position information about the pathological finding on an ultrasonic image. The anatomical position information may include information for specifying the position of a pathological finding in a relationship between a plurality of structures included in the ultrasonic image. The processor 120 may display an anatomical position of the pathological finding through at least one of the graphical indicator, the text indicator, and a color highlight.

For example, referring to FIG. 16, the processor 120 may display the text indicator indicating the order of structures (D21a, D21b, D21c, D21d, ...) of constituting the spine in order to display that a filar cyst is positioned near L3 on the ultrasonic image and that a conus medullaris is detected near L2-L3 which is an abnormal position. According to an embodiment, by displaying the text indicator indicating the order of the structures of constituting the spine, it may be intuitively conveyed to the user that the conus medullaris is positioned beyond a range of the normal position and that the filar cyst is positioned near L3.

Depending on the various embodiments, dotted lines, solid lines, dashed lines, colored lines, etc., may be used in order to clearly display the position of the filar cyst.

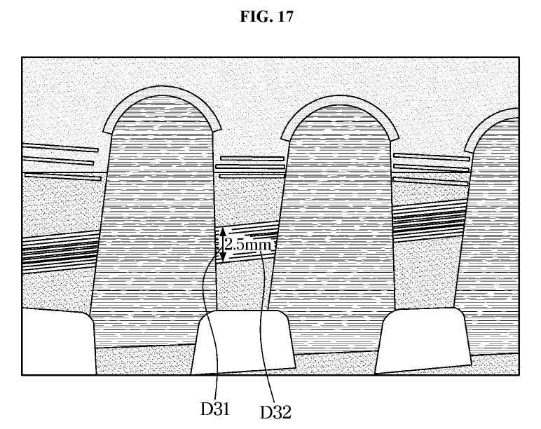

FIG. 17 is a view illustrating an example of a cross-sectional ultrasonic image of the spine displayed with numerical value information about the pathological finding according to an embodiment.

The processor 120 may display at least one numerical value of a pathological finding included in the ultrasonic image through at least one of the graphical indicator and the text indicator.

When there is a plurality of ultrasonic images, the processor 120 may display at least one numerical value of a pathological finding included in each of the plurality of ultrasonic images through at least one of the graphical indicator and the text indicator on each ultrasonic image.

The numerical value of a pathological finding may include a numerical value about a size of the pathological finding. For example, the numerical value of a pathological finding may include a diameter, area, or volume of the pathological finding or at least a portion of the pathological finding.

The processor 120 may automatically calculate the numerical value of a pathological finding and display the numerical value on an ultrasonic image. For example, the processor 120 may recognize a border of the pathological finding upon recognizing the pathological finding, and automatically calculate a numerical value based on the recognized border.

Additionally, the processor 120 may produce a numerical value based on the user designating a specific pathological finding or at least a portion of a pathological finding on the ultrasonic image. The processor 120 may receive annotation button input of the user for designating a specific pathological finding on the ultrasonic image from input interface 170. The processor 120 may calculate and display a numerical value based on user input.

The text indicator may provide a numerical value of a pathological finding in text format. The processor 120 may display the numerical value directly near the pathological finding displayed on a screen of the display 140, or may display the numerical value in a separate text box or data panel. For example, referring to FIG. 17, the processor 120 may display a value (D31) about the thickness of the spinal cord filum terminale according to the thickened spinal cord filum terminale of the pathological finding, near the pathological finding.

The graphic indicator may be provided in the form of a graph, color display, histogram, or infographic in order to visually represent the numerical value of the pathological finding. For example, referring to FIG. 17, the processor 120 may display a caliper marker or arrow (D32) near the pathological finding in order to display a measured length or width of the pathological finding. According to various embodiments, the processor 120 may display a borderline on the ultrasonic image in order to highlight the border of the pathological finding.

The processor 120 may provide comprehensive numerical values of pathological findings by combining the text indicator and the graphic indicator. For example, referring to FIG. 17, the processor 120 may display an arrow with a value about the thickness of the spinal cord filum terminale in order to display the measured length or width of the pathological finding.

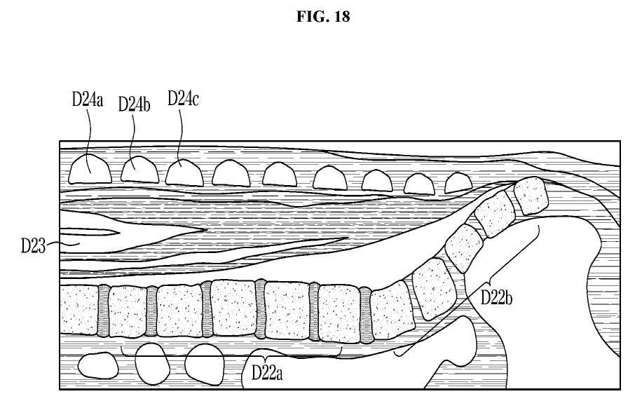

Additionally, the processor 120 may display the graphic indicator on the ultrasonic image by overlay in order to display the anatomical position information about the pathological finding. In this regard, a detailed description will be provided with reference to FIGS. 18 to 20.

FIGS. 18 to 20 illustrate examples of cross-sectional ultrasonic images of the spine in which pathological findings included in ultrasonic images and a plurality of structures or a plurality of distinguishing regions positioned around the pathological findings are distinguished and displayed according to an embodiment.

The processor 120 may identify borders between a pathological finding included in the ultrasonic image and a plurality of structures or a plurality of distinguishing regions positioned around the pathological finding, in order to display the anatomical position information about the pathological finding. Accordingly, the processor 120 may display the pathological finding and the plurality of structures or the plurality of distinguishing regions through the graphic indicator, text indicator, and a color highlight so that the pathological finding and the plurality of structures or the plurality of distinguishing regions are distinguished based on the identified borders.

When there is a plurality of ultrasonic images, the processor 120 may identify borders between a pathological finding included in each of the plurality of ultrasonic images and a plurality of structures or a plurality of distinguishing regions positioned around the pathological finding. Accordingly, the processor 120 may display the pathological finding and the plurality of structures or the plurality of distinguishing regions on each ultrasonic image through at least one of the graphic indicator, text indicator, and color highlight so that the pathological finding and the plurality of structures or the plurality of distinguishing regions are distinguished based on the identified borders.

In an embodiment, the user may intuitively detect pathological findings on ultrasonic images. The user may also visually clearly represent a relationship between structures and pathological findings that are difficult to identify with ultrasonic images alone. Accordingly, it may be used as an important tool in establishing diagnosis and treatment plans.

The plurality of structures positioned around the pathological finding may include anatomical or physiological structures positioned near the pathological finding. For example, the plurality of structures positioned around the pathological finding may include at least one of a normal tissue, organ, and vascular structure in proximity to the pathological finding.

The plurality of distinguishing regions positioned around the pathological finding may include a specific spatial region preset centered about the pathological finding. That is, the graphic indicator, text indicator, or color highlight may be utilized to distinguish between pathological findings from other parts on the ultrasonic image.

FIG. 18 is a view illustrating another example of an ultrasonic image displayed with the anatomical position information about the pathological finding according to an embodiment.

According to an embodiment, the processor 120 may display the graphic indicator imaging a plurality of structures or a plurality of distinguishing regions based on the identified border. The displaying of the graphic indicator imaging a plurality of structures or a plurality of distinguishing regions may include displaying a body marker. The processor 120 may image the pathological finding and the plurality of structures or the plurality of distinguishing regions through the graphic indicator so that the pathological finding and the plurality of structures or the plurality of distinguishing regions are clearly distinguished based on the identified borders. For example, referring to FIG. 18, the processor 120 may image various anatomical structures (e.g., lumbar spine (D22a), sacrum (D22b), conus medullaris (D23), spinous processes (D24a, D24b, D24c, etc.)) and display them on the ultrasonic image by overlay.

For example, referring to FIG. 18, the processor 120 may display information that the end of the conus medullaris (D23) is positioned near lumbar spine 2 and lumbar spine 3 through body markers for a lumbar spine (D22a) and a sacrum (D22b).

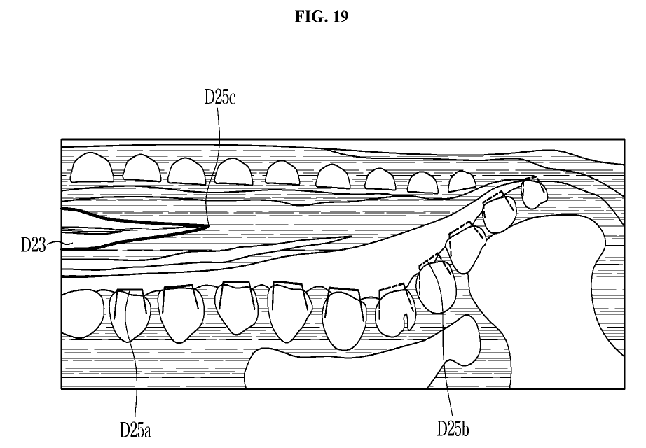

FIG. 19 is a view illustrating another example of a cross-sectional ultrasonic image of the spine displayed with the anatomical position information about the pathological finding according to an embodiment.

According to an embodiment, the processor 120 may display the identified borders by boundary overlay in different colors. The processor 120 may display the borders between a pathological finding, surrounding tissues, structures, or a specific distinguishing region with different colors so that the pathological finding and the plurality of structures or the plurality of distinguishing regions are clearly distinguished based on the identified borders. An original ultrasonic image itself is often expressed in black and white (or grayscale), but the processor 120 may generate color overlay in a method of overlaying a borderline on this black and white image. In this case, the processor 120 may determine colors for distinguishing the borders between the pathological finding, surrounding tissues, structures, or specific distinguishing region based on the user input received through the input interface 170. For example, referring to FIG. 19, the processor 120 may display a first color line (D25a) indicating a border of the lumbar spine, a second color line (D25b) indicating a border of the sacrum, and a third color line (D25c) indicating a border of the conus medullaris on the ultrasonic image by overlay. Accordingly, referring to FIG. 19, the processor 120 may display the information that the end of the conus medullaris (D23) is positioned near lumbar spine 2 and lumbar spine 3 through the first color line (D25a) and the third color line (D25c).

In this case, the first color line (D25a), the second color line (D25b), and the third color line (D25c) may be the same or different colors. Additionally, the processor 120 may determine a color of each color line based on the user input received through the input interface 170. Accordingly, the user may intuitively understand a relationship between the pathological finding and the surrounding anatomical structures or distinguishing regions.

FIG. 20 is a view illustrating another example of a cross-sectional ultrasonic image of the spine displayed with the anatomical position information about the pathological finding according to an embodiment.

According to an embodiment, the processor 120 may display a plurality of structures or a plurality of distinguishing regions with different colors based on the identified borders by color overlay. The processor 120 may display a pathological finding, surrounding tissues, structures, or specific distinguishing regions with different colors by overlay so that the pathological finding and the plurality of structures or the plurality of distinguishing regions are clearly distinguished based on the identified borders. For example, referring to FIG. 20, the processor 120 may display a conus medullaris region with a preset color by overlay. The processor 120 may display neural lines together when displaying the conus medullaris region with the preset color by overlay. In this case, the nerve lines may be displayed in a different color than the color overlay corresponding to the conus medullaris region.

Referring to FIG. 20, the processor 120 may display the information that the end of the conus medullaris is positioned near lumbar spine 2 and lumbar spine 3 using the preset color. Additionally, the processor 120 may not only display the conus medullaris region in the preset color (D26a) by overlay, but also display the neural lines in a different color than the color overlay corresponding to the conus medullaris region.

The indication of anatomical position information about one pathological finding on an ultrasonic image through the color overlay has been described above with reference to FIG. 20. However, according to various embodiments, the processor 120 may display anatomical position information about each of a plurality of pathological findings through color overlay. For example, each of the plurality of pathological findings may be color overlaid in a different color (e.g., a first color, a second color, etc.). In this case, the first color and the second color may be the same or different colors. Additionally, the processor 120 may determine a color of each color overlay based on the user input received through the input interface 170. Accordingly, the user may intuitively understand the relationship between the pathological finding and the surrounding anatomical structures or distinguishing regions.

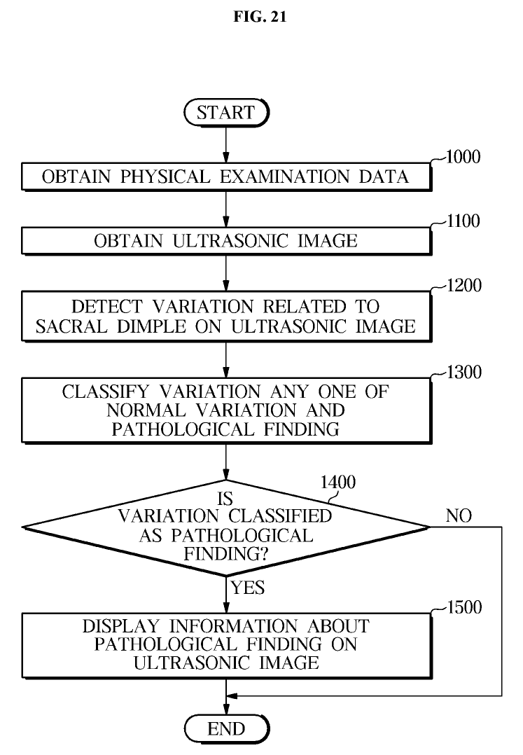

FIG. 21 is a control flowchart of an ultrasonic diagnostic apparatus according to an embodiment.

According to an embodiment, the processor 120 may obtain physical examination data (1000). The physical examination data may correspond to any data obtained by the user through visual observation or physical examination (e.g., palpation, auscultation, etc.). According to an embodiment, the physical examination data may include clinical features (hereinafter referred to as 'clinical features') related to the sacral dimple. That is, at least part of the physical examination data highly related to the typical sacral dimple or the atypical sacral dimple may correspond to the clinical features.

According to one embodiment, the processor 120 may obtain an ultrasonic image (1100). For example, the processor 120 may transmit an ultrasonic wave from a probe 20f to an object and receive an echo signal reflected from the ultrasonic wave to obtain at least one ultrasonic image. The at least one ultrasonic image may include a cross-sectional ultrasonic image of the spine. For example, the at least one ultrasonic image may include at least one of a long-axis spine cross-sectional ultrasonic image (longitudinal Image) and a short-axis spine cross-sectional ultrasonic image (transverse Image).

The processor 120 may detect a variation related to the sacral dimple in the obtained ultrasonic image (1200). In this case, the variation related to the sacral dimple may include all differences related to the sacral dimple appearing on the ultrasonic image. For example, the variation may include a tumor, cyst, inflammation, abnormal reflection (echo) pattern, or difference in the shape of an organ or tissue.

The processor 120 may determine the sacral dimple type of the object based on the obtained physical examination data. In this case, the sacral dimple type may be determined as typical or atypical type. The typical sacral dimple may be referred to as a simple sacral dimple. Additionally, the sacral dimple type may be determined as one of type 1, type 2, or type 3. In this case, the typical sacral dimple may correspond to type 1. The atypical sacral dimple may include type 2 and type 3. In other words, the atypical sacral dimple may be classified as type 2 or type 3 depending on the degree of the pathological state thereof.

The processor 120 may adjust the identification sensitivity based on the determined sacral dimple type in detecting the variations. The adjusting of the identification sensitivity may include adjusting at least one factor that determines how sensitively a variation is detected on the ultrasonic image.

For example, the processor 120 may decrease the identification sensitivity when the sacral dimple type is determined to be a typical sacral dimple. The decreasing of the identification sensitivity may include detecting only relatively large and distinct abnormal symptoms and ignoring small variations.

For example, the processor 120 may increase the identification sensitivity when the sacral dimple type is determined to be an atypical sacral dimple. The increasing of the identification sensitivity may include monitoring even small or subtle variations.

For example, the processor 120 may adjust the identification sensitivity depending on the degree of the pathological state thereof when the sacral dimple type is determined to be an atypical sacral dimple. For example, the processor 120 may increase the identification sensitivity more when the sacral dimple type is determined to be type 3 than when the sacral dimple type is determined to be type 2.

According to an embodiment, the processor 120 may classify the detected variation as any one of a normal variation and a pathological finding (1300). The normal variation is a change that may differ from person to person anatomically or physiologically, but may correspond to a difference appearing within the normal range. That is, when the detected variation is classified as the normal variation, it is not a disease or abnormal state, and therefore treatment may not be necessary. On the other hand, the pathological finding may correspond to the case in which the variation detected on the ultrasonic image is outside the normal range. That is, when the detected variation is classified as the pathological finding, it may be clinically significant and may require additional examination or medical intervention.

The processor 120 may classify the variation detected on the ultrasonic image as a pathological finding based on the anatomical criteria. In this case, the anatomical criteria for classifying the variation may be pre-stored in the memory 150.

According to an embodiment, the processor 120 may determine whether the variation is classified as the pathological finding (1400). The processor 120 may display information about the pathological finding on the obtained ultrasonic image (1500) when the variation is classified as the pathological finding (YES in 1400). The information about the pathological finding may include at least one of the name information about the pathological finding, predicted disease information related to the pathological finding, anatomical position information about the pathological finding, or numerical information about the pathological finding.

For example, the processor 120 may display the name information about the pathological finding and/or predicted disease information related to the pathological finding using the graphic indicator or the text indicator.

For example, the processor 120 may display multiple predicted disease information related to the predetermined pathological finding on an ultrasonic image. The at least one of the multiple predicted disease information to be displayed on the ultrasonic image in relation to the predetermined pathological finding may be preset and stored in the memory 150. The processor 120 may display the preset predicted disease information as a fixed value at the preset position when the variation detected on the ultrasonic image is classified as the predetermined pathological finding. In this case, the preset predicted disease information may correspond to a predicted disease most frequently diagnosed when the pathological finding is detected. According to various embodiments, the processor 120 may change and/or add to the preset predicted disease information based on the user input received through the input interface 170.

For example, the processor 120 may display the anatomical position of the pathological finding using at least one of the graphical indicator, the text indicator, and the color highlight.

For example, the processor 120 may display at least one numerical value of the pathological finding included in the ultrasonic image through at least one of the graphical indicator and the text indicator.

For example, the processor 120 may display the graphic indicator on the ultrasonic image by overlay in order to display the anatomical position information about the pathological finding.

For example, the processor 120 may identify borders between the pathological finding included in the ultrasonic image and a plurality of structures or a plurality of distinguishing regions positioned around the pathological finding, in order to display the anatomical position information about the pathological finding. Accordingly, the processor 120 may display the pathological finding and the plurality of structures or the plurality of distinguishing regions using at least one of the graphic indicator, text indicator, and color highlight so that the pathological finding and the plurality of structures or the plurality of distinguishing regions are distinguished based on the identified borders. The processor 120 may display the graphic indicator imaging the plurality of structures or the plurality of distinguishing regions based on the identified borders. The processor 120 may display the identified borders by boundary overlay in different colors. The processor 120 may display the plurality of structures or the plurality of distinguishing regions with different colors based on the identified borders by color overlay.

A control method of an ultrasonic diagnostic apparatus according to an embodiment may include: obtaining physical examination data about a sacrum skin lesion of an object from a user; obtaining an ultrasonic image of the object; detecting a variation related to a sacral dimple on the ultrasonic image; classifying the variation as any one of a normal variation and a pathological finding; and displaying information about the pathological finding on the ultrasonic image based on the variation being classified as the pathological finding.

The detecting of the variation related to the sacral dimple on the ultrasonic image may include determining a sacral dimple type of the object based on the physical examination data and adjusting an identification sensitivity based on the determined sacral dimple type.

The adjusting of the identification sensitivity based on the determined sacral dimple type may include decreasing the identification sensitivity based on the sacral dimple type being determined to be a typical sacral dimple.

The adjusting of the identification sensitivity based on the determined sacral dimple type may include increasing the identification sensitivity based on the sacral dimple type being determined to be an atypical sacral dimple.

The displaying of the information about the pathological finding on the ultrasonic image based on the variation being classified as the pathological finding may include displaying at least one of name information about the pathological finding included in the ultrasonic image, predicted disease information related to the pathological finding, anatomical position information about the pathological finding, or numerical information about the pathological finding.

The displaying of the at least one of the name information about the pathological finding and the predicted disease information related to the pathological finding may include displaying at least one of the name information about the pathological finding and the predicted disease information related to the pathological finding using at least one of a graphic indicator and a text indicator.

The displaying of the anatomical position information about the pathological finding may include displaying the anatomical position information about the pathological finding using at least one of the graphic indicator, the text indicator, or a color highlight.

The displaying of the numerical information about the pathological finding may include displaying the numerical information about the pathological finding using at least one of the graphic indicator and the text indicator.

The displaying of the anatomical position information about the pathological finding using at least one of the graphic indicator, the text indicator, or the color highlight may include identifying borders between the pathological finding included in an ultrasonic image and a plurality of structures or a plurality of distinguishing regions positioned around the pathological finding, and displaying the pathological finding and the plurality of structures or the plurality of distinguishing regions using at least one of the graphic indicator, the text indicator, or the color highlight so that the pathological finding and the plurality of structures or the plurality of distinguishing regions are distinguished based on the identified borders.

The displaying of the pathological finding and the plurality of structures or the plurality of distinguishing regions using the graphic indicator so that the pathological finding and the plurality of structures or the plurality of distinguishing regions are distinguished based on the identified borders may include displaying the pathological finding and the plurality of structures or the plurality of distinguishing regions using the graphic indicator imaging the plurality of structures or the plurality of distinguishing regions based on the identified borders.

The displaying of the pathological finding and the plurality of structures or the plurality of distinguishing regions using the color highlight so that the pathological finding and the plurality of structures or the plurality of distinguishing regions are distinguished based on the identified borders may include displaying the identified borders in different colors by boundary overlay.

The displaying of the pathological finding and the plurality of structures or the plurality of distinguishing regions using the color highlight so that the pathological finding and the plurality of structures or the plurality of distinguishing regions are distinguished based on the identified borders may include displaying the plurality of structures or the plurality of distinguishing regions in different colors by color overlay based on the identified borders.

An ultrasonic diagnostic apparatus according to an embodiment may include: a probe configured to transmit an ultrasonic signal to an object and receive information about an echo signal reflected from the object; and a main body including a display, an input interface configured to receive user input, and a processor configured to obtain physical examination data about a sacrum skin lesion of the object from the input interface, obtain an ultrasonic image of the object based on the information about the echo signal obtained from the probe, detect a variation related to a sacral dimple on the ultrasonic image, classify the variation as any one of a normal variation and a pathological finding, and control the display to display information about the pathological finding on the ultrasonic image based on the variation being classified as the pathological finding.

The processor may be configured to determine a sacral dimple type of the object based on the physical examination data and adjust an identification sensitivity based on the determined sacral dimple type.

The processor may be configured to control the display to display information about the normal variation on the ultrasonic image based on the variation being classified as the normal variation.

The information about the normal variation may include at least one of name information about the normal variation, anatomical position information about the normal variation, or numerical information about the normal variation.

The processor may be configured to display at least one of name information about the pathological finding included in the ultrasonic image, predicted disease information related to the pathological finding, anatomical position information about the pathological finding, or numerical information about the pathological finding based on the variation being classified as the pathological finding.

The processor may be configured to display at least one of the name information about the pathological finding and the predicted disease information related to the pathological finding using at least one of a graphic indicator and a text indicator.

The processor may be configured to display the anatomical position information about the pathological finding using at least one of the graphic indicator, the text indicator, or a color highlight.

The processor may be configured to display the numerical information about the pathological finding using at least one of the graphic indicator and the text indicator.

As is apparent from the above, according to an aspect of the disclosure, various abnormal findings related to an atypical sacral dimple are displayed on an ultrasonic image, thereby enabling accurate diagnosis of spinal and neurological abnormalities, which can contribute to early treatment of newborns and infants.

According to an aspect of the disclosure, the quality of a medical service and the marketing effectiveness of a hospital can be improved simultaneously by providing intuitive indication and explanation so that an examiner and a guardian can easily understand the examination result.

According to an aspect of the disclosure, accurate diagnosis is possible regardless of the skill of the examiner by automatically indicating a normality or an abnormality on the ultrasonic image, and an examination time can be shortened and efficiency can be significantly improved.

However, effects that can be achieved by an ultrasonic diagnostic apparatus and a control method thereof according the disclosure are not limited to the effects mentioned above, and other effects not mentioned will be clearly understood by those skilled in the art to which the disclosure belongs from the above description.

The disclosed embodiments may be implemented in the form of a recording medium storing instructions executable by a computer. The instructions may be stored in the form of program code, and when executed by a processor, a program module may be created to perform the operations of the disclosed embodiments. The recording medium may be implemented as a computer-readable recording medium.

The computer-readable recording medium includes any type of recording medium in which instructions readable by the computer are stored. For example, the recording medium may include a read only memory (ROM), a random access memory (RAM), a magnetic tape, a magnetic disk, a flash memory, an optical data storage device, and the like.

In addition, the computer-readable recording medium may be provided in the form of a non-transitory storage medium. Herein, the 'non-transitory storage medium' simply means that it is a tangible device and does not contain signals (e.g., electromagnetic waves), and this term does not distinguish between cases where data is semi-permanently stored in a storage medium and cases where data is stored temporarily. For example, the 'non-transitory storage medium' may include a buffer where data is temporarily stored.

According to an embodiment, methods according to various embodiments disclosed in this document may be provided and included in a computer program product. The computer program product is a commodity and may be traded between sellers and buyers. The computer program product may be distributed in the form of a machine-readable recording medium (e.g., compact disc read only memory (CD-ROM)), or may be distributed (e.g., downloaded or uploaded) online, through an application store (e.g., Play Store™) or directly between two user devices (e.g., smartphones). In the case of online distribution, at least a portion of the computer program product (e.g., a downloadable app) may be at least temporarily stored or created temporarily in the machine-readable recording medium, such as the memory of a manufacturer server, an application store server, or a relay server.

The foregoing has illustrated and described specific embodiments. However, it should be understood by those of skilled in the art that the disclosure is not limited to the above-described embodiments, and various changes and modifications may be made without departing from the technical idea of the disclosure described in the following claims.

What is claimed is:

1. A control method of an ultrasonic diagnostic apparatus comprising:
obtaining physical examination data about a sacrum skin lesion of an object from a user;
obtaining an ultrasonic image of the object;
detecting a variation related to a sacral dimple on the ultrasonic image;
classifying the variation as any one of a normal variation and a pathological finding; and
displaying information about the pathological finding on the ultrasonic image based on the variation being classified as the pathological finding.

2. The control method according to claim 1, wherein
the detecting of the variation related to the sacral dimple on the ultrasonic image comprises: determining a sacral dimple type of the object based on the physical examination data; and adjusting an identification sensitivity based on the determined sacral dimple type.

3. The control method according to claim 2, wherein
the adjusting of the identification sensitivity based on the determined sacral dimple type comprises decreasing the identification sensitivity based on the sacral dimple type being determined to be a typical sacral dimple.

4. The control method according to claim 2, wherein
the adjusting of the identification sensitivity based on the determined sacral dimple type comprises increasing the identification sensitivity based on the sacral dimple type being determined to be an atypical sacral dimple.

5. The control method according to claim 1, wherein
the displaying of the information about the pathological finding on the ultrasonic image based on the variation being classified as the pathological finding comprises displaying at least one of name information about the pathological finding included in the ultrasonic image, predicted disease information related to the pathological finding, anatomical position information about the pathological finding, or numerical information about the pathological finding.

6. The control method according to claim 5, wherein
the displaying of the at least one of the name information about the pathological finding and the predicted disease information related to the pathological finding comprises displaying at least one of the name information about the pathological finding and the predicted disease information related to the pathological finding using at least one of a graphic indicator and a text indicator.

7. The control method according to claim 5, wherein
the displaying of the anatomical position information about the pathological finding comprises displaying the anatomical position information about the pathological finding using at least one of a graphic indicator, a text indicator, or a color highlight.

8. The control method according to claim 5, wherein
the displaying of the numerical information about the pathological finding comprises displaying the numerical information about the pathological finding using at least one of a graphic indicator and a text indicator.

9. The control method according to claim 7, wherein
the displaying of the anatomical position information about the pathological finding using at least one of the graphic indicator, the text indicator, or the color highlight comprises: identifying borders between the pathological finding included in an ultrasonic image and a plurality of structures or a plurality of distinguishing regions positioned around the pathological finding; and displaying the pathological finding and the plurality of structures or the plurality of distinguishing regions using at least one of the graphic indicator, the text indicator, or the color highlight so that the pathological finding and the plurality of structures or the plurality of distinguishing regions are distinguished based on the identified borders.

10. The control method according to claim 9, wherein
the displaying of the pathological finding and the plurality of structures or the plurality of distinguishing regions using the graphic indicator so that the pathological finding and the plurality of structures or the plurality of distinguishing regions are distinguished based on the identified borders comprises displaying the pathological finding and the plurality of structures or the plurality of distinguishing regions using the graphic indicator imaging the plurality of structures or the plurality of distinguishing regions based on the identified borders.

11. The control method according to claim 9, wherein
the displaying of the pathological finding and the plurality of structures or the plurality of distinguishing regions using the color highlight so that the pathological finding and the plurality of structures or the plurality of distinguishing regions are distinguished based on the identified borders comprises displaying the identified borders in different colors by boundary overlay.

12. The control method according to claim 9, wherein
the displaying of the pathological finding and the plurality of structures or the plurality of distinguishing regions using the color highlight so that the pathological finding and the plurality of structures or the plurality of distinguishing regions are distinguished based on the identified borders comprises displaying the plurality of structures or the plurality of distinguishing regions in different colors by color overlay based on the identified borders.

13. An ultrasonic diagnostic apparatus comprising:
a probe configured to transmit an ultrasonic signal to an object and receive information about an echo signal reflected from the object; and
a main body comprising a display, an input interface configured to receive user input, and a processor configured to obtain physical examination data about a sacrum skin lesion of the object from the input interface, obtain an ultrasonic image of the object based on the information about the echo signal obtained from the probe, detect a variation related to a sacral dimple on the ultrasonic image, classify the variation as any one of a normal variation and a pathological finding, and control the display to display information about the pathological finding on the ultrasonic image based on the variation being classified as the pathological finding.

14. The ultrasonic diagnostic apparatus according to claim 13, wherein
the processor is configured to determine a sacral dimple type of the object based on the physical examination data and adjust an identification sensitivity based on the determined sacral dimple type.

15. The ultrasonic diagnostic apparatus according to claim 13, wherein
the processor is configured to control the display to display information about the normal variation on the ultrasonic image based on the variation being classified as the normal variation.

16. The ultrasonic diagnostic apparatus according to claim 15, wherein
the information about the normal variation comprises at least one of name information about the normal variation, anatomical position information about the normal variation, or numerical information about the normal variation.

17. The ultrasonic diagnostic apparatus according to claim 13, wherein
the processor is configured to display at least one of name information about the pathological finding included in the ultrasonic image, predicted disease information related to the pathological finding, anatomical position information about the pathological finding, or numerical information about the pathological finding based on the variation being classified as the pathological finding.

18. The ultrasonic diagnostic apparatus according to claim 17, wherein
the processor is configured to display at least one of the name information about the pathological finding and the predicted disease information related to the pathological finding using at least one of a graphic indicator and a text indicator.

19. The ultrasonic diagnostic apparatus according to claim 17, wherein
the processor is configured to display the anatomical position information about the pathological finding using at least one of a graphic indicator, a text indicator, or a color highlight.

20. The ultrasonic diagnostic apparatus according to claim 17, wherein
the processor is configured to display the numerical information about the pathological finding using at least one of a graphic indicator and a text indicator.
